(19) United States
(12) Patent Application Publication
(10) Pub. No.: US 2010/0185042 A1
(43) Pub. Date: Jul. 22, 2010

(54) CONTROL AND COORDINATION OF TRANSCRANIAL MAGNETIC STIMULATION ELECTROMAGNETS FOR MODULATION OF DEEP BRAIN TARGETS

(76) Inventors: M. Bret Schneider, Portola Valley, CA (US); David J. Mishelevich, Playa del Rey, CA (US)

Correspondence Address: SHAY GLENN LLP, 2755 CAMPUS DRIVE, SUITE 210, SAN MATEO, CA, 94403, US

(21) Appl. No.: 12/701,395

(22) Filed: Feb. 5, 2010

Related U.S. Application Data
(63) Continuation-in-part of application No. 12/185,544, filed on Aug. 4, 2008.
Application No. 12/185,544 is a continuation-in-part of application No. PCT/US2008/075575, filed on Sep. 8, 2008.
Application No. PCT/US2008/075575 is a continuation-in-part of application No. PCT/US2008/075583, filed on Sep. 8, 2008.
Application No. PCT/US2008/075583 is a continuation-in-part of application No. PCT/US2008/075824, filed on Sep. 10, 2008.
Application No. PCT/US2008/075824 is a continuation-in-part of application No. PCT/US2008/081048, filed on Oct. 24, 2008.
(60) Provisional application No. 60/954,018, filed on Aug. 5, 2007.
(60) Provisional application No. 60/970,532, filed on Sep. 7, 2007.
(60) Provisional application No. 60/970,534, filed on Sep. 7, 2007.
(60) Provisional application No. 60/975,177, filed on Sep. 26, 2007.
(60) Provisional application No. 60/970,945, filed on Sep. 8, 2007.
(60) Provisional application No. 60/971,211, filed on Sep. 10, 2007.
(60) Provisional application No. 60/982,141, filed on Oct. 24, 2007.

(51) Int. Cl. A61N 2/02

(52) U.S. Cl. 600/13

(57) ABSTRACT

Described herein are devices and method for control and coordination of TMS electromagnets for modulation of deep brain targets. For example, described herein are methods and devices for stimulating neural structures within the brain using multi-coil arrays. Also described herein are devices and methods that relate generally to the focusing of magnetic fields generated by electromagnets used for Transcranial Magnetic Stimulation. Devices and methods relating generally to the focusing of magnetic fields generated by electromagnets used for Transcranial Magnetic Stimulation are also described, as well as devices and methods that relate generally to moving and positioning electromagnets generating magnetic fields used for Transcranial Magnetic Stimulation. Finally, also described are devices and methods that relate generally to control of moving, positioning, and activating electromagnets generating magnetic fields used for Transcranial Magnetic Stimulation.

### CROSS REFERENCE TO RELATED APPLICATIONS

This application claims priority as a continuation-in-part of U.S. patent application Ser. No. 12/185,544, titled “MONOPHASIC MULTI-COIL ARRAYS FOR TRANCRANIAL MAGNETIC STIMULATION,” filed on Aug. 4, 2008, which claims priority to U.S. Provisional Patent Application Ser. No. 60/954,018, titled “MONOPHASIC MULTI-COIL ARRAYS FOR TRANCRANIAL MAGNETIC STIMULATION” filed on Aug. 5, 2007. This application is also a continuation-in-part of PCT International Patent Application Serial No. PCT/US2008/075575, titled “FOCUSING MAGNETIC FIELDS WITH ATTRACTOR MAGNETS AND CONCENTRATOR DEVICES,” filed on Sep. 8, 2008 (published as WO 2009/033144), which claims priority to U.S. Provisional Patent Applications: No. 60/970,532, titled “FOCUSING MAGNETIC FIELDS WITH CONCENTRATION DEVICES,” filed on Sep. 7, 2007; No. 60/970,534, titled “FOCUSED MAGNETIC FIELDS USING ATTRACTOR MAGNETS,” filed on Sep. 7, 2007; and No. 60/975,177, titled “FOCUSING MAGNETIC FIELDS WITH ATTRACTOR MAGNETS AND CONCENTRATOR DEVICES,” filed on Sep. 26, 2007. This application is also a continuation-in-part of PCT International Patent Application Serial No. PCT/US2008/075583, titled “PITCH, ROLL, AND YAW MOTIONS FOR ELECTROMAGNET ARRAYS,” filed on Sep. 8, 2008 (published as WO 2009/033150), which claims priority to U.S. Provisional Patent Application Ser. No. 60/970,945, titled “PITCH, ROLL, AND YAW MOTIONS FOR ELECTROMAGNET ARRAYS,” filed on Sep. 8, 2007. This application is also a continuation-in-part of PCT International Patent Application Serial No. PCT/US2008/075824, titled “AUTOMATED MOVEMENT OF ELECTROMAGNETS TRACKING ECCENTRICITY OF THE HEAD,” filed on Sep. 10, 2008 (published as WO 2009/036040), which claims priority to U.S. Provisional Patent Application Ser. No. 60/971,211, titled “AUTOMATED MOVEMENT OF ELECTROMAGNETS TRACKING ECCENTRICITY OF THE HEAD,” filed on Sep. 10, 2007. This application is also a continuation-in-part of PCT International Patent Application Serial No. PCT/US2008/081048, titled “INTRA-SESSION CONTROL OF TRANSCRANIAL MAGNETIC STIMULATION,” filed on Oct. 24, 2008 (published as WO 2009/055634), which claims priority to U.S. Provisional Patent Application Ser. No. 60/982,141, titled “INTRA-SESSION CONTROL OF TRANSCRANIAL MAGNETIC STIMULATION,” filed on Oct. 24, 2007. All of these patent applications are herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The devices and methods described herein relate generally to the use of one or more (and particularly arrays) Transcranial Magnetic Stimulation electromagnets to stimulate the brain, and particularly to stimulate deep brain target region of the brain for therapeutic purposes.

### BACKGROUND OF THE INVENTION

Concurrent use of more than one magnetic stimulation coil can be used to improve depth of stimulation within a brain, and to help control the location of a deep area of stimulation, as described in M. B. Schneider et al., filed Apr. 9, 2004, U.S. patent application Ser. No. 10/821,807, and Mishelevich et al., filed May 5, 2006 U.S. patent application Ser. No. 11/429,504. However, presently available rTMS pulse generator units are limited in their ability to provide the optimal signal to such a coil array. Most magnetic nerve stimulators in use today are biphasic or polyphasic, for example the Magstim Rapid2 (Magstim Ltd., Wales, UK). Electrically efficient, they are well suited to sustained, rapid pulse trains needed for producing enduring brain modulation, for example depression treatments. However, the complexity of the polyphasic waveform, meeting the complexity of the nervous system frequently yields inconsistent results. In the case of multiple coil stimulation, there is an increased chance that one waveform phase from one coil could diminish or cancel a concurrent phase from another coil Monophasic magnetic stimulators provide more predictable neurostimulation effects, and they are well known in the art. For example, the Magstim 2002 (Magstim Ltd, Wales, UK), and the MagPro X100 with MagOption by the Dantec division of Medtronic (Copenhagen, Denmark), which generates pulse shapes including biphasic, monophasic, and half-sine. Electrically inefficient, these machines are not capable of sustained, rapid pulse trains. The output of these devices is always to a single output to a coil in which all loop portions receive the same electrical waveform.

The prior art does not provide any means for coordinating pulse phase, timing polarity or strength between more than one coil.

Also, Transcranial Magnetic Stimulation (TMS) has been previously delivered from electromagnets positioned at the side of the head, the top of the head, or somewhere in between the side and the top of the head. Generally speaking, a single or double standard TMS coil placed on a patient's scalp and operated at a power level at, or slightly above, a patient's motor threshold will directly active neurons from the cortical crowns to the bottom of the cortical gyri-a depth of about 1-3 cm. Using this approach, deeper structures (herein referred to as “subcortical”, even when these deeper areas are histologically layered in nature) are activated only secondarily through intracerebral neural connections. Conventional approaches typically do not reach greater depths (for example, the cingulate gyms, the insula and other subcortical structures). Deep brain modulation cannot be accomplished by simply turning up the power of the stimulating electromagnet, because the intervening tissue, for example superficial cortex, will be over-stimulated, causing undesired side effects such as seizures.

Positive outcomes for treatment of depression refractory to drug treatment have been demonstrated with repetitive Transcranial Magnetic Stimulation (rTMS, Avery et al., 2005). rTMS works indirectly, because the superficial stimulation of the dorsolateral pre-frontal cortex is carried by nerve fibers to the deeper cingulate gyms. More effective therapy of depression and treatment of a number of other conditions such as chronic pain, addiction, obesity, and obsessive compulsive disorder would be possible with focused brain stimulation capable of reaching depths below the cortex. Devices for providing deep brain stimulation with Transcranial Magnetic Stimulation are described in Schneider and Mishelevich, U.S. patent application Ser. No. 10/821,807 and Mishelevich and Schneider, U.S. patent application Ser. No. 11/429,504. Whether superficial or deep stimulation is being employed, focusing the applied magnetic field during TMS has the potential to improve clinical results. In particular, the ability to stimulate at depth could be facilitated by shaping the profile of the magnetic field of one or more primary stimulating electromagnets, thereby focusing their magnetic fields and more preferentially stimulating a given targeted neural structure.

The magnetic fields used for Transcranial Magnetic Stimulation determine both the depth and size of the region of stimulation. Thus, a more focused magnetic field would be capable of stimulating an area that is also more tightly focused, and may be better controlled by the TMS system.

Both deep-brain stimulation and direct stimulation of deeper brain regions could benefit from improved focusing of the magnetic field of the primary stimulating electromagnets. Described herein are systems, methods and devices for improving the focus of the primary electromagnets used for Transcranial Magnetic Stimulation allowing enhanced stimulation of targeted neural structures.

Stereotactic Transcranial Magnetic Stimulation (stereotactic TMS) concentrates the effects of applied magnetic fields at a target deep in the brain. This must be done without over-stimulating the superficial brain (cortex) and causing negative side effects such as seizures. One important strategy for such deep-brain TMS stimulation is to fire the electromagnets so that the electromagnetic energy from each electromagnet passing through a given non-target point of the cortex is minimized. For example, a given steady repetition rate of firing a single electromagnet or set of electromagnets can overwhelm the non-target superficial cortex through which the electromagnet energy must pass on the way to the deeper target.

One way to avoid over-stimulating these superficial non-target regions (which may elicit undesirable side-effects such as seizures) is to move the electromagnets around a subject's head while focusing their electromagnetic energy on deep targets, to avoid frequent firing through the same non-target portion of the cortex. For example, U.S. patent application Ser. No. 10/821,807 to Schneider and Mishelevich describes using a gantry to which the electromagnets are attached that allows the positions of the electromagnets around the head to be moved (e.g., rotated).

An alternative approach is to this method is describe herein. In particular, described herein are TMS systems, devices and methods configured for limited rotational movements (including oscillatory movements) in of either the framework (e.g., gantry) or individual electromagnets in one or more of pitch, roll and yaw.

Transcranial Magnetic Stimulation (TMS) typically involves the application of electromagnetic fields to one or more target brain regions in order to excite or inhibit the target brain regions. For example, a single or double standard TMS coil placed on a patient's scalp and operated at a power level at, or slightly above, a patient's motor threshold will directly active neurons from the cortical crowns to the bottom of the cortical gyri- a depth of about 1-3 cm. Using this approach, deeper structures (herein referred to as “sub-cortical”, even when these deeper areas are histologically layered in nature) are activated only secondarily through intracerebral neural connections. Conventional approaches typically do not reach greater depths (for example, the cingulate gyms, the insula and other sub-cortical structures). Deep brain modulation cannot be accomplished by simply turning up the power of the stimulating electromagnet, because the intervening tissue, for example superficial cortex, will be over-stimulated, causing undesired side effects such as seizures.

Positive outcomes for treatment of depression refractory to drug treatment have been demonstrated with rTMS (repetitive Transcranial Magnetic Stimulation (Avery et al., 2005). rTMS works indirectly because the superficial stimulation of the dorsolateral pre-frontal cortex is carried by nerve fibers to the deeper cingulate gyms. More effective therapy of depression and treatment of a number of other conditions such as chronic pain, addiction, obesity, and obsessive compulsive disorder would be possible with focused brain stimulation at depth. U.S. patent application Ser. No. 10/821,807 describes one variation of a device for providing deep brain stimulation with Transcranial Magnetic Stimulation.

In general, controlling the position of the TMS electromagnet relative to the subject's head (and therefore the target brain regions) is critical to any application of TMS, including deep brain modulation as described in U.S. patent application Ser. No. 10/821,807. For example, Schneider and Mishelevich, in U.S. Patent Application Publication No. 2005/0228209 describe the rotation of one or more electromagnets used in Transcranial Magnetic Stimulation of the brain around a subject's head to achieve TMS stimulation of deep structures. One embodiment described is the spinning of an electromagnet around the cranium on a movable gantry.

When moving or rotating TMS electromagnets about a subject's head, it may be particularly helpful to use a trajectory following the contour of the subject's head, rather than simply a circular, elliptical or other orbit. A circular orbit is inefficient from the energy delivery standpoint: magnetic field strength falls off rapidly as it leaves the face of a coil. In the case of transcranial magnetic stimulation, a circular orbit would mean that much energy delivered from the coil would never enter the brain of the user, as it would decay within the air spaces between the coil face and the sides of the head.

Although an electromagnet could be positioned closer or farther from a central target location in the brain by a robot arm moving closer or farther as required to avoid hitting the patient's head. However, currently described robotic arm systems (see, e.g., Fox et al. US 2003/0050527) are overly complex and expensive, and would require complicated control systems in order to maintain appropriate spacing between the electromagnet and the subject's head. Thus, it would be preferable to have the radial movement accomplished automatically using mechanical means, which may be more robust, reliable and lower-cost.

Transcranial Magnetic Stimulation (TMS) of targets within the brain or other parts of the nervous system may modulate neural activity, and may be used to treat a variety of disorders, behaviors and indications. For example, positive outcomes for treatment of depression refractory to drug treatment have been demonstrated with rTMS (repetitive Transcranial Magnetic Stimulation, Avery et al., 2005). rTMS is believed to work indirectly because the superficial stimulation of the dorsolateral pre-frontal cortex is carried by nerve fibers to the deeper cingulate gyms. TMS of both superficial and deep-brain regions may also be used to treat other disorders or conditions, such as acute or chronic pain, addiction, obesity, and obsessive compulsive disorder (OCD).

TMS therapies for many of these disorders is likely to be successful only if stimulation of deep-brain target regions can be achieved. Deep brain targets are of particular interest for TMS, but practical deep-brain TMS has been difficult to achieved, because stimulation at depth must be performed without over-stimulating superficial tissues. Recently, Schneider and Mishelevich, U.S. patent application Ser. No. 10/821,807 and Mishelevich and Schneider, U.S. patent application Ser. No. 11/429,504, have described methods for achieving TMS stimulation of deep brain regions without over stimulating (or in some cases even stimulating) regions superficial to the deep brain region.

In operation, TMS may be performed on a patient by a medical professional that operates the system. The medical professional adjusts the system, e.g., the magnet(s), to target one or more brain regions, and will typically use brain scans or maps of the particular patent's brain (e.g., using MRI, or other imaging techniques). In addition to positioning the magnet(s), the practitioner may also set the intensity (e.g., power) and frequency of firing of the TMS electromagnets. Thus, the targeting and stimulation levels are usually done in an open-loop system, without substantial functional feedback from the patient or patient being treated. This type of control does not allow functional feedback, and may be less accurate and also less effective than a system that would somehow directly confirm adequate stimulation of the appropriate brain region necessary for achieving therapeutic effect. Described herein are systems and methods that allow direct patient feedback based on acute effects of TMS that have are correlated with the target therapy.

In contrast with the direct patient feedback described herein, simple indirect patient feedback is known. For example, indirect physiologic feedback during a TMS session includes brain imaging during the course of a TMS procedure. For example, EEG and fMRI instrumentation have been employed. Use of the EEG domain is described in Ives and Pascual-Leone, U.S. Pat. Nos. 6,266,256 and 6,571,123. Use of the fMRI domain are described in Ives, et al. U.S. Pat. No. 6,198,958 and Bohning and George, U.S. patent application Ser. No. 10/991,129. These employ interleaving of TMS and fMRI. George et al., U.S. patent application Ser. No. 10/521,373 describe using TMS to prevent a patient from deceiving the user, but not for alleviating a condition; the process is also used in conjunction with fMRI.

Motor feedback based on stimulation parameters has also been described (Riehl, U.S. Pat. No. 7,104,947), but is not applicable to the conditions addressed herein. Fox and Lancaster, U.S. Pat. No. 7,087,008 teach a robotic system for positioning TMS coils involving PET scanning to locate the target, but the system does not use direct patient-reported feedback. Tanner (U.S. Pat. Nos. 6,830,544 and 7,239,910) has described presentation of stimuli (e.g., optical, auditory, or olfactory) in the usual way and then adjusting applied TMS to reproduce the same sensations as closely as possible, in some cases using passive markers for navigation. Tanner et al., in U.S. Pat. No. 7,008,370, describe matching coordinates of a simulation model generated from MRI data with a model of the TMS induction device, positioning the electromagnet on the head, and stimulating using the electromagnet to get a response (such as an EMG response in the forearm of the patient) indicating the mapping. These mapping methods are used as part of a mapping method for investigating only normal functions and do not deal with treatment.

While the above-described approaches can be useful, they are not applicable in ambulatory settings where the vast numbers of patients will be treated. Further, all of these methods operate by inference, based on generalization of treatment of brain regions, and assume that the desired therapeutic effect will be follow TMS of the patient's brain region, rather than assess the effects of the TMS directly. What is needed is a mechanism to obtain direct immediate patient-reported feedback from the patient and make intra-session adjustments accordingly.

Although patient-reported feedback has been applied with some success in other treatment types, such as implantable electrode stimulation, it has not been applied to TMS therapies. For example, Mayberg and Lazano have previously documented patient-reported immediate feedback in deep brain stimulation using implanted electrodes. They describe patients who reported immediate lifting of depressive systems while undergoing deep brain stimulation using electrodes inserted in the brain tissue.

The methods, devices and systems for TMS described herein all include patient feedback and/or control of the TMS stimulation in a manner that may allow enhanced accuracy and efficacy over previous TMS therapy methods.

### SUMMARY OF THE INVENTION

This application is divided, merely for the sake of convenience into five parts. Part I describes methods and devices for stimulating neural structures within the brain using multi-coil arrays. Part II describes devices and methods that relate generally to the focusing of magnetic fields generated by electromagnets used for Transcranial Magnetic Stimulation. Part III describes devices and methods relating generally to the focusing of magnetic fields generated by electromagnets used for Transcranial Magnetic Stimulation. Part IV describes devices and methods that relate generally to moving and positioning electromagnets generating magnetic fields used for Transcranial Magnetic Stimulation. Part V describes devices and methods that relate generally to control of moving, positioning, and activating electromagnets generating magnetic fields used for Transcranial Magnetic Stimulation.

The variations and embodiments described in each part may be used separately or in combination. In particular, elements describe in any of these parts may be used (in part or in whole) with elements from any of the other parts.

For example, in part I, a device is described in which a biphasic or polyphasic electrical discharge from a magnetic nerve stimulation or rTMS machine is split into two separate monophasic pulses. These separate pulses are then sent to two separate coils. The method provides for the coordination of pulse polarity, phase, timing, and strength between multiple magnetic stimulation coils. The goal is to optimize the manner in which multiple coils may be used synergistically to control the activity of underlying neural tissue.

In one embodiment a biphasic electrical pulse is passed through a high-power bridge rectifier, with the two outputs of which power the positive pole of one double coil and the negative pole of the other double coil, while the remaining pole of each of two double coils are both held to ground. The electrical pulses through each of the coils is thereby monophasic, and the induced magnetic field, is at least substantially monophasic.

In an alternative embodiment, the two poles of a biphasic electrical pulse generator are passed through high-power diodes, the positive pole of one double coil and the negative pole of the other double coil, while the remaining pole of each of two double coils are both held to ground. The electrical pulses through each of the coils is thereby monophasic, and the induced magnetic field, is at least substantially monophasic.

In another alternative embodiment, a biphasic electrical pulse source has its poles connected with opposite sides of a high-power bridge rectifier. The outputs from the remaining sides of the bridge rectifier are passed to the positive and negative poles of one double coil. The electrical pulses through each of the coils is thereby monophasic, and the induced magnetic field, is at least substantially monophasic.

Pulse generation devices that produce such pulses are commercially available such as the Magstim Rapid stimulator by Magstim LTD (Wales, UKCoils that are useful within the context of the present invention are commercially available, for example the 70 mm double coil (Magstim LTD (Wales, UK).

In one embodiment, coils may be situated substantially next to one another such that their magnetic fields are directed in a substantially similar direction if the coils are arranged in the same polarity, and a substantially opposite direction if the coils are arranged with opposite polarities. In another embodiment, the coils may be situated substantially opposite one another, such that their magnetic fields are directed in a substantially opposite direction if the coils are situated with the same polarity, and substantially the same if the coils are situated in opposite polarity.

In part II, methods, devices and systems for controlling, focusing and/or modifying magnetic fields are described which may be appropriate for use with Transcranial Magnetic Stimulation (TMS), including repetitive Transcranial Magnetic Stimulation (rTMS). In particular, described herein are devices, systems, and methods including one or more magnetic field modifiers such as attractor magnets and/or concentration devices.

As used herein, an attractor magnet is typically a secondary magnet positioned separately from the primary TMS magnet(s) whose magnetic field the attractor magnet is configured to modify. An attractor magnet may be isolated from the primary TMS electromagnet(s) whose field it is configured to modify. In particular the attractor magnet may be physically isolated, meaning that it may not be directly connected to a primary electromagnet. In some variations, the attractor magnet is separately maneuverable from the primary electromagnet, though it may be connected to the same gantry, framework, etc. as the primary electromagnet(s). For example, an attractor magnet may be positioned opposite from a primary TMS magnet and configured to direct, focus, or otherwise enhance the electromagnetic field emitted by the TMS magnet, which may aid in delivering deeper, more effective Transcranial Magnetic Stimulation (TMS). Typically, TMS involves uses a large electromagnet placed near the side of the patient's head to provide electromagnetic simulation. For the purposes of this document, the primary TMS (e.g., large) electromagnet is herein referred to as a “primary magnet,” “primary electromagnet” or a “main electromagnet”. The secondary magnets described herein, which may be located separately from the primary magnet but can be activated simultaneously or synchronously (in the case of active secondary magnets) with the primary magnet. For example, one or more attractor magnets may be located on the side of a patient's head opposite from the primary TMS magnet(s), or in the mouth or nasal cavity, and may act as active sink for the magnetic field from the magnet to help focus the field.

The secondary magnets (or “attractor magnets”) may be of opposite polarity in a phase-complementary manner at any given time within the course of synchronized TMS discharges from the primary TMS magnet(s). Consequently, the attractor magnet may draw in the primary electromagnet's magnetic field. Alternatively, the primary magnet may be described as drawing out or focusing the magnetic field of the attractor magnet(s). Although many of the attractor magnets described herein are active electromagnets (e.g., to which current may be applied to generate an electromagnetic field), attractor magnets may also be configured as permanent magnets, and the similar principles of operation may be applied to permanent magnets in addition to (or instead of) electromagnets. Thus, in some variations, an attractor magnet is (or includes) a permanent magnet. In some variations the attractor magnet is an electromagnet (also referred to as an active magnet).

Because the magnetic fields produced by TMS magnets have complex, 3-dimensional field strength profiles, the term “opposite” polarity may be relative. Consequently, the primary TMS magnet and an attractor magnet do not need to be positioned at 180 degrees with respect to one another. For example, a primary magnet and an attractor magnet may face 90 degrees relative to one another, and still produce the attractor effect as herein described. In some variations the attractor magnet may be moved relative to the TMS magnet (or vice versa). In general, the attractor magnet may be positioned so that the effect of the attractor magnet on the magnetic field of the TMS magnet is predictable.

One or more attractor magnets may be used simultaneously to modify the magnetic field of a primary magnet. In addition, attractor magnets may be used with one or more other magnetic field modification devices, including magnetic concentrators. A magnetic concentrator typically includes a shaped region of high magnetic permeability that may focus an externally applied magnetic field. One or more magnetic concentrators may help focus on neural-tissue targets in the brain (or spinal cord) during TMS.

A magnetic concentrator typically includes one or more regions of high magnetic permeability. The material of high magnetic permeability may be shaped or formed into a shape to guide, concentrate, or limit the pathway of the magnetic field through the magnetic concentrator. A region of high magnetic permeability may be formed or high magnetic permeability alloys such as “Mu metal”, which may draw and thus concentrate magnetic fields in these regions. The magnetic concentrators described herein (for use with TMS systems) can be disposable, or including one or more disposable components. A disposable concentrator can be customized to the appropriate shape for a given patient at a selected anatomical location. For example, a magnetic concentrator may be configured for use in a patient's oral cavity, nasal cavity, sinus cavities, external ear canals or on the surface of the body, and may be customized to fit a particular patient, or may be generic to patients (or categories of patients).

In general, the attractor magnets and magnetic concentrators (or “TMS concentrators”) described herein may be used alone, or in any appropriate combinations. Systems including one or more of these magnetic modifiers may also include a control unit. The control unit may be part of the TMS system control unit, and may include logic to determine and account for the effect of the magnetic field modifier on the TMS magnet(s). The control unit may also control the attractor magnet; for example, the activation of the attractor magnet may be controlled by the control unit. The control unit (or a separate unit) may also position, suggest positions, and/or confirm the positioning of the attractor magnet(s) and/or magnetic concentrator(s).

Part III describes systems, devices and method for Transcranial Magnetic Stimulation (TMS) in which the TMS electromagnets are configured to move in a pitch motion, a roll motion, a yaw motion, or two or three of those in combination. Moving the TMS electromagnet(s) in pitch, roll and/or yaw during treatment may avoid over-stimulating structures and causing undesirable side effects such as seizures.

In general the systems described herein include a frame assembly and one or more TMS electromagnets secured to the frame assembly, as well as actuator and associated control mechanisms configured to move the TMS electromagnet(s) in pitch, roll and/or yaw. In some variations, the TMS electromagnets included in the system may be individually moved, so that each TMS electromagnet (or some subset or sub-combination of TMS electromagnets) may be independently moved (e.g., rotated) in pitch, roll or yaw. For example, each TMS electromagnet may be connected to one or more actuators and associated control mechanisms for moving it in pitch, roll and/or yaw. In other variations, all of the TMS electromagnets (e.g., an entire array) may be moved in pitch, roll and yaw as a unit. For example, the frame assembly including an entire array of electromagnets may be moved in pitch, roll and/or yaw.

The rotational motion of the TMS electromagnets in pitch, roll and yaw may mean that the overall translational movement of the TMS electromagnet relative to the subject's head may be slight. For example, moving individual (or subsets) of electromagnets in pitch, roll and yaw may result in minor rotations of the electromagnets relative to other portions of the system.

As described herein, the rotational movements of the TMS electromagnets may be driven by one or more actuators that may be controlled by one or more controllers. An actuator may include any appropriate mechanism, including rotary actuators, linear actuators, magnetic actuators, geared actuators, incorporating step-motors, servo motors or the like, or any device that will provide the required motion. Any appropriate controller may be used. For example, a controller may include one or more of hardware, software, firmware or some combination thereof. For example, a controller may execute control logic for determining (e.g., calculating) the motion, including the timing of the motion, of one or more TMS electromagnets. One or more sensors, including position sensors, may be used. Sensors may provide the controller with positional information on the frame, TMS electromagnets, and/or the subject's body (e.g., head). Any appropriate sensor may be used, including optical, mechanical, etc.

For example, described herein are Transcranial Magnetic Stimulation systems for stimulating a subject's neuronal system that include: a frame assembly; at least one Transcranial Magnetic Stimulation electromagnet supported by the frame; and a plurality of actuators (such as motors), wherein the actuators are configured to move the Transcranial Magnetic Stimulation electromagnet in roll, pitch and yaw. The systems may also include a controller communicating with the plurality of actuators, wherein the controller is configured to control movement of the Transcranial Magnetic Stimulation electromagnet in roll, pitch and yaw.

In some variations, the systems include a plurality of Transcranial Magnetic Stimulation electromagnets supported by the frame, wherein the plurality of actuators are configured to move the plurality of Transcranial Magnetic Stimulation electromagnets in roll, pitch and yaw. For example, the plurality of actuators may be configured to move the plurality of Transcranial Magnetic Stimulation electromagnets by moving the frame assembly in roll, pitch and yaw. Alternatively, each of the Transcranial Magnetic Stimulation electromagnets may be configured to be independently moveable in roll, pitch and yaw with respect to the other Transcranial Magnetic Stimulation electromagnets.

Also describe herein are Transcranial Magnetic Stimulation systems for stimulating a subject's neuronal system that include: a frame assembly; an array of Transcranial Magnetic Stimulation electromagnets supported by the frame; and a plurality of actuators, wherein the actuators are configured to move the array of Transcranial Magnetic Stimulation electromagnets in roll, pitch and yaw. The system may also include a controller communicating with the plurality of actuators and configured to control movement of the Transcranial Magnetic Stimulation electromagnet in roll, pitch and yaw. As mentioned, the plurality of actuators may be configured to simultaneously move the entire array of Transcranial Magnetic Stimulation electromagnets in roll, pitch and yaw by moving the frame assembly, and/or the Transcranial Magnetic Stimulation electromagnets may be configured to be independently moveable in roll, pitch and yaw with respect to the other Transcranial Magnetic Stimulation electromagnets.

Also described herein are Transcranial Magnetic Stimulation systems for stimulating a subject's neuronal system including: a frame assembly; a plurality of Transcranial Magnetic Stimulation electromagnets supported by the frame; a plurality of actuators; and a controller communicating with the plurality of actuators, wherein the Transcranial Magnetic Stimulation electromagnets is configured to be moved in roll, pitch and yaw by the actuators, so that each Transcranial Magnetic Stimulation electromagnet or a subset of the Transcranial Magnetic Stimulation electromagnets may be moved relative to the other Transcranial Stimulation electromagnets.

Methods of applying TMS by rotating the TMS electromagnets in roll, pitch and/or yaw are also described herein. For example, described herein are Transcranial Magnetic Stimulation methods for stimulating a neuronal target tissue, the method comprising: positioning a frame including at least one Transcranial Magnetic Stimulation electromagnet around a subject's head; and moving the Transcranial Magnetic Stimulation electromagnet in roll, pitch and/or yaw.

The step of moving the Transcranial Magnetic Stimulation electromagnet may include moving the frame to which the Transcranial Magnetic Stimulation electromagnet(s) is attached, or moving the Transcranial Magnetic Stimulation electromagnet(s) relative to another Transcranial Magnetic Stimulation electromagnet included on the frame.

In general these systems may be configured to move the TMS electromagnets either before during or after activation of the electromagnets. For example, a treatment method may include repetitively firing the TMS electromagnet and moving one or more TMS electromagnets in roll, pitch and/or yaw during or between firing. The methods described herein may also include the step of activating the Transcranial Magnetic Stimulation electromagnet prior to moving the Transcranial Magnetic Stimulation electromagnet. In general, the method may include the step of activating one or more actuators to move the Transcranial Magnetic Stimulation electromagnet.

In some variations, the step of moving the Transcranial Magnetic Stimulation electromagnet comprises moving the Transcranial Magnetic Stimulation electromagnet in roll pitch and/or yaw relative to the frame.

Also described herein are Transcranial Magnetic Stimulation methods for stimulating a neuronal target tissue, that include the steps of: positioning a frame including a plurality of Transcranial Magnetic Stimulation electromagnet around a subject's head; and moving one or a subset of the Transcranial Magnetic Stimulation electromagnets in roll, pitch and/or yaw relative to the other Transcranial Magnetic Stimulation electromagnet(s). In some variations, this method may include activating one or more of the Transcranial Magnetic Stimulation electromagnets prior to the step of moving one or a subset of the Transcranial Magnetic Stimulation electromagnets.

Part IV describes devices, systems and methods for moving one or more electromagnets around a subject's head by having the electromagnets connected to a gantry and adjusting the distance between the patient's head and the electromagnet by adjusting the position of the electromagnets relative to the gantry. Thus, as an electromagnet is rotated around the subject's head, the electromagnet is moved outwards or inwards relative to the gantry to maintain a desired distance from the subject's head.

In general, the Transcranial Magnetic Stimulation (TMS) systems described herein include a gantry and a TMS actuator module that includes a TMS electromagnet and an actuator for moving the TMS electromagnet relative to the gantry. The actuator may be configured to move the TMS electromagnet in and/or out relative to the gantry (e.g., closer or farther from the patient's head), or it may be configured to move the TMS electromagnet around a track on the gantry; for non-round gantries, this will move the magnet closer and further from the subject's head. In this embodiment, the entire gantry may also be rotated around the subject's head so that the magnet may be positioned over the appropriate region of the head.

For example, described herein are TMS systems including: a gantry configured to at least partially encircle a patient's head; and a TMS actuator module connected to the gantry, wherein the TMS actuator module comprises a TMS electromagnet and an actuator for moving the TMS electromagnet in or out relative to the gantry. The TMS actuator module is further configured to move the TMS electromagnet to adjust the distance between the TMS electromagnet and the surface of the patient's head.

The gantry may be a frame or track. The gantry is generally configured to at least partially surround the patient's head, and may be configured so that the gantry can be positioned over the patient's head, or the patient's head may be placed into a fixed (or securable) gantry. The gantry may be circular, semi-circular, oval, semi-oval, or any other appropriate shape. Although planar gantries are shown herein, other shapes, including gantries that allow the TMS actuator modules to be positioned at different heights as well as different radial positions relative the subject's head may be used.

In general, the TMS actuator modules described herein may include one or more TMS electromagnets, and may be either movably connected to the gantry, or they may be fixed to the gantry. For example, the TMS actuator modules may be moved around the patient's head by riding on the gantry as a ‘track’. In some variations, the entire gantry may be moved (e.g., rotated) around the patient's head.

As mentioned, the TMS actuators described herein typically include a TMS electromagnet. Any appropriate TMS electromagnet (which may also be referred to as a “primary TMS electromagnet”) may be used. For example, the TMS electromagnet may be a 70 mm double-coil electromagnet may be used, such as the 70 mm double-coil configuration from Magstim (Model 9925, Wales, UK). Other TMS electromagnets having different configurations may also be used.

In some variations, the actuator included as part of the TMS actuator module is configured for moving the TMS electromagnet in and/or out relative to the gantry. This actuator may be a linear or uniaxial actuator that is configured to drive the TMS electromagnet position either forward or backward (e.g., in/out relative to the gantry to which the module is attached). Any appropriate actuator may be used, as described herein, including mechanical (e.g., threaded, geared, worm-screw, etc.), pneumatic, piezoelectric, etc. Solenoid-type actuators may also be used, and may be shielded or isolated from the TMS electromagnet to avoid interference with the TMS electromagnet field and operation.

In some variations, the distance between the TMS electromagnet and the subject is adjusted by moving both the TMS electromagnets around the gantry and by moving the gantry around the patient's head. For example, when a patient's head is positioned in the center of an oval gantry, moving the TMS electromagnet closer to the short axis of the oval will shorten the distance between the TMS electromagnet and the center of the oval; conversely, moving the TMS electromagnet closer to the long axis increases the distance to the center of the oval. Thus, moving the TMS electrode (which may be part of a TMS actuator module) around the gantry changes the radial position relative to the center of the gantry and therefore the patient, while moving the entire gantry may position the TMS electrode relative to the region of the patient's head. The combined motions of the gantry rotation and rotation of the TMS actuator module around the gantry, may be used to control the distance between the subject and the TMS electromagnet. In this embodiment, the TMS actuator module typically includes one or more TMS electromagnets and at least one gantry/magnet actuator configured to move the TMS electromagnet(s) around the gantry.

The systems described herein may also include one or more controllers configured to determine the position of the TMS actuator module and to instruct the actuator of the TMS actuator module to adjust position of the TMS electromagnet relative to a patient's head. A controller may be part of an overall system controller (or sub-system controller), and may execute control logic to determine the position and/or position adjustments to apply. Controllers may include hardware, software, firmware, or any other appropriate structures necessary to perform the function described herein. Controllers may be analog or digital, and may receive input (including feedback/feedforward input) from one or more detectors and/or sensors, particularly position detectors.

The system may include any appropriate detector, including a position detector. For example, a position detector may be configured to determine the position of the TMS actuator module on the gantry. Gantry/actuator position detectors may be particularly useful in variations in which the module moves along the gantry. In some variations, the position of the module on the gantry may be used to indicate how the TMS actuator should be controlled. For example, the controller may include pre-set or pre-determined instructions for positioning the TMS electromagnet relative to the gantry at certain gantry positions. Thus, the instructions may be based on a general head shape relative to the gantry shape, or they may be determined by mapping the gantry position relative to a subject's head after positioning the gantry relative to the subject's head (positioning the gantry relative to the subject's head may mean either placing the gantry near the subject's head, or placing the subject's head in/near the gantry). In some variations, the gantry includes markings, marks or other indicators that indicate where a module is on the gantry; a detector may sense these indicators to determine position.

One or more position detectors for detecting the orientation of the gantry relative to the patient (gantry/patient detectors) may also be used. These detectors may be particularly helpful in variations in which the entire gantry is rotated.

In some variations, the detector is a position detector configured to determine the position of the TMS electromagnet relative to a patient's head. These detectors (e.g., “patient/electromagnet detectors”) may also be referred to as head-magnet detectors. A head position detector may include any appropriate sensor, such as an optical sensor, etc.

In some variations, the system includes a plurality of TMS actuator modules, wherein each TMS actuator module comprises a TMS electromagnet and an actuator for moving the TMS electromagnet in or out relative to the gantry. Any appropriate number of modules may be included, and a single controller may be used to control all or a subset of the modules, or multiple controllers may be used.

In variations in which the TMS actuator module is configured to move along the gantry, the module may include one or more a gantry/magnet actuators configured to move the TMS actuator module along the gantry. In some variations the gantry includes a track that is geared, and the gantry/magnet actuator may move by engaging the gears.

In some variations, the system includes one or more tilt actuators as part of the TMS actuator module. A tilt actuator is typically configured to adjust the angle of the TMS electromagnet relative to a patient's head. Tilt actuators may allow slight rotation (which may be limited) in one or two axis (e.g., the axes in the plane or face of the TMS facing the subject's head) so that the angle of the TMS magnet's emitted field relative to the patient's head can be controlled. Tilt actuators may be mechanical, piezo, or any other appropriate actuator. The controller may also be configured to control the tilt actuator(s).

Also described herein are TMS system comprising: a gantry; a TMS actuator module on the gantry, wherein the TMS actuator module comprises a TMS electromagnet, and a uniaxial actuator for moving the TMS electromagnet in or out relative to the gantry; at least one position detector configured to determine the position of the TMS electromagnet relative to a patient's head; and a controller configured to determine the position of the TMS electromagnet relative to the patient's head based on input from the position detector, and to instruct the TMS actuator module to adjust the distance between the TMS electromagnet and the surface of the patient's head based on the determined position.

As mentioned above, the TMS actuator module may be configured to move along the gantry and comprise a gantry/magnet actuator configured to move the TMS actuator module along the gantry. Alternatively, the TMS actuator module may be secured to the gantry.

Methods for TMS stimulation, including methods of positioning a TMS electromagnet relative to a subject' head are also described. In general, these methods include the steps of moving a TMS actuator module around the subject's head and actuating the TMS actuator to move the TMS electromagnet along one axis (e.g., in/out) to move it closer or further from the subject's head, based on the position of the module relative to the subject's head.

For example, described herein are TMS methods comprising: moving a TMS actuator module along a gantry, wherein the TMS actuator module comprises a TMS electromagnet and an actuator configured to move the TMS electromagnet towards or away from a patient's head relative to the gantry; and adjusting the distance between the TMS electromagnet and the patient's head using the actuator.

In any of the systems and methods described herein, the distance between the subject's head and the TMS electromagnet may be kept at a constant distance. For example, the step of adjusting the distance between the TMS electromagnet and the patient's head may comprise maintaining a relatively constant distance between the TMS electromagnet and the patient's head as the TMS actuator module is moved along the gantry.

The methods may also include the step of activating the TMS electromagnet to apply electromagnetic field to neuronal target. In some variations, the targets may be deep brain targets; in some variations, the targets are cortical or superficial targets.

The method may also include the step of determining the position of the TMS electromagnet relative to the patient's head. For example, the method may include the step of sensing distance between TMS electromagnet and the subject's head. In some variations, the step of adjusting the distance between the TMS electromagnet and the patient's head includes determining the distance between the TMS electromagnet and the subject's head and controlling the actuator to adjust the distance of the TMS electromagnet based on this distance.

Also described herein are Transcranial Magnetic Stimulation (TMS) methods including the steps of: positioning a gantry relative to a subject's head; moving a TMS actuator module along a gantry, wherein the TMS actuator module comprises a TMS electromagnet and an actuator configured to move the TMS electromagnet towards or away from a patient's head relative to the gantry; determining the position of the TMS electromagnet relative to the patient's head; adjusting the distance between the TMS electromagnet and the patient's head using the actuator; and activating the TMS electromagnet to apply an electric field to a neuronal target. As mentioned, the step of adjusting the distance between the TMS electromagnet and the patient's head comprises maintaining a relatively constant distance between the TMS electromagnet and the patient's head as the TMS actuator module is moved along the gantry. In some variations, the system includes feedback from the sensors that is configured to respond sufficiently quickly so the patient will not be struck by the device.

In addition to systems and methods for moving a TMS electromagnet around a subject's head, the principles described herein for moving around a subject's head may also be used for moving other devices (including energy sources and/or sensors) around a subject's head. For example, described herein are transcranial tracking systems comprising: a gantry configured to at least partially encircle a patient's head; and an actuator module connected to the gantry, wherein the actuator module comprises an energy source and an actuator for moving the energy source in or out relative to the gantry; wherein the actuator module is further configured to move the energy source to adjust the distance between the energy source and the surface of the patient's head. The energy source may be selected from the group consisting of: light, radio frequency, acoustic, and, thermal energy sources, or any other appropriate energy source.

Also described herein are transcranial tracking system, the system comprising: a gantry configured to at least partially encircle a patient's head; and an actuator module connected to the gantry, wherein the actuator module comprises a sensor and an actuator for moving the sensor in or out relative to the gantry; wherein the actuator module is further configured to move the sensor to adjust the distance between the energy source and the surface of the patient's head.

Also described herein are TMS systems and methods including a gantry configured as a track along which one or more TMS electromagnets may move or be positioned. In such variations, the gantry may be shapeable or configurable to a patient's head shape. For example, the gantry may be customizable to fit the contours of a patient's head. Thus, the gantry may be flexible and/or shapeable or may include links or hinge regions allowing its shape around the subject's head to be spaced an appropriate distance (e.g., a fixed distance) from the subject's head.

In operation, such a customizable gantry may be pre-fit to a specific patient prior to TMS or other operation. For example, in some methods, the gantry may be made flexible or movable, and adjusted to be spaced around a subject's head. After (or during) this fitting step, the gantry may be locked into position. For example, the gantry may includes a track made of lockable links (e.g., each 1 mm or less), that can be configured to the head profile, then tensioned or otherwise locked into position. TMS treatment may then be performed using the shaped track. The shape of the gantry may be provided to a controller for the TMS system. For example, the relative position along the gantry corresponding to various patient head anatomy may be correlated prior to treatment and provided to the controller, which can use this information to control the application of TMS. During treatment with the customized gantry, it is not necessary to move the TMS electromagnet in/out relative to the gantry, as described for other variations. Thus, these variations may not require an additional TMS actuator module, although in some variations it may be beneficial to include such a module, or a module modified to include tilt actuators and or an actuator for moving the TMS electrode around the subject's head.

Part V describes systems and methods for treating a patient with TMS, and particularly deep brain TMS, in which the patient has a least limited control of one or more TMS parameters, such as the position of the TMS magnet(s), the intensity of the TMS stimulation (e.g., applied magnetic field), or the frequency of the TMS stimulation. This patient feedback is based on the patient's acute experience during the TMS stimulation, which may be provoked by a stimulus, or unprovoked.

In general, the patient undergoing the TMS therapy alters one or the TMS parameters (e.g., using one or more inputs) based on one or more acute responses to the TMS procedure. Thus, any of the methods described herein may include feedback from the patient to alter a parameter based on the patient's experience. For example, if the patient is being treated for pain, and the patient does not experience a cessation or lessening of acute pain during TMS treatment, the patient may change the treatment (e.g., move the TMS electromagnet or increase the stimulation intensity or increase the stimulation frequency), until the pain is lessened. In some situations direct and immediate feedback from the patient may be triggered by alleviation of the condition being treated. For example, when treating depression, stimulation with TMS deep within the brain (e.g., using techniques such as Schneider and Mishelevich, U.S. patent application Ser. No. 10/821,807 and Mishelevich and Schneider, U.S. patent application Ser. No. 11/429,504), immediate relief (e.g., positive feelings) may be experienced. Indirect stimulation of the cingulate gyms by superficial rTMS (repetitive Transcranial Magnetic Stimulation) has already demonstrated immediate increased blood flow with Positive Emission Tomography (PET) using oxygen or glucose-mediated agents.

Acute responses may be triggered or provoked by a stimulus correlated with the disorder, disease or behavior being treated. In some treatments the patient experience tied to the patient's feedback may be related to the disorder being treated. For example, during treatment the patient may experience an immediate symptom reduction (e.g., acute pain, drug addiction). Some conditions to which superficial or deep TMS are applicable may have no immediate acute demonstrable patient-reported effect during the session (e.g., obesity). In such cases, a proxy or surrogate acute response experienced during treatment may be used to trigger patient feedback, and the patient may be instructed or trained to respond to the surrogate. For example, treatment side effects including stimulation site pain, visual disturbances and induced motor activity may be present during the course of a typical treatment session, and one or more of these side effects may be correlated with a desired treatment region. For example, when treating pain or attempting to effect anesthesia/analgesia, the cessation of stimulation site pain may trigger feedback by the patient.

The stimulation applied to any target, including the targets identified herein, may be either up- or down-regulating stimuli. For example, “up regulation” in a particular brain region may mean stimulation at a frequency of about 5 Hz or greater within the target region. Similarly, “down-regulation” of a target region may refer to stimulation at a rate of 1 Hz or less.

In some variations, the acute experience used by the patient to control the TMS therapy may be triggered by a stimulus during (or immediately before) application of the TMS therapy. For example, if treating a disorder such as obsessive compulsive disorder (OCD), the patient may be exposed to a stimulus would normally cause anxiety (e.g., a soiled garment, an unpleasant image, etc.). Other conditions with potential for immediate feedback include addiction and addictive behaviors, in which the patient may be exposed to a stimulus that would normally trigger an emotional response, such as drugs, cigarettes, alcohol or food. This triggering stimulus may be applied during or before TMS treatment, and the patient may then experience an acute reduction in the effect.

For patients having chronic conditions with an acute equivalent (e.g., chronic pain), checking the patient's response to an acute version could permit inferences related to the chronic version to be used for treatment planning. For example, consider a patient with chronic pain. Using our invention either there will be immediate relief or not; in the first case, the given patient will get immediate relief from his or her chronic pain with suitably adjusted TMS. In the second case, If immediate relief from chronic pain does not occur because the chronic pain condition will require repetitive treatments to bring relief, the approach would be to cause the patient to have an acceptable level of acute pain (say by applying a noxious substance such as capsaicin pepper extract) as a surrogate for chronic pain, adjust the TMS parameters to get maximum relief for the acute pain, and use then those same parameters for subsequent TMS treatments of the chronic pain. While the chronic-pain pathway response may not exactly mirror that for acute pain, it would be an excellent place to start.

Although patient feedback/control during the TMS therapy is typically experiential, or based on the reported experience of the patient, it may also (or alternatively) be controlled by one or more involuntary, unconscious, and/or physiological patient responses. For example, successful TMS treatment may cause an involuntary or physiological response that is not recognized by the patient, such as increase or decrease in heart rate, blood pressure, respiratory rate, etc. This type of ‘involuntary’ patient feedback may also be detected by the system, and may be used to modify the treatment. In some variations, the system may prevent false or erroneous reporting of conscious or volitional feedback by requiring both unconscious and conscious feedback. For example, if treating pain, the system may allow the patient to continue to adjust one or more parameter during TMS treatment (patient control feedback), as long as an ‘unconscious’ patient feedback does not indicate successful treatment (e.g., change in heart rate, blood pressure, etc., indicating alleviation in pain). Alternatively, the unconscious or involuntary patient feedback may be used to select the parameter controlled by the patient or the magnitude of the patient control.

For example, described herein are patient-configurable Transcranial Magnetic Stimulation (TMS) methods that allows a patient to dynamically modify the TMS while a TMS procedure is being performed. In some variations, the methods include the steps of: applying Transcranial Magnetic Stimulation to a first site in the patient's brain, at a first magnetic field intensity and a first stimulation frequency; changing one or more of the site, intensity or the frequency of the TMS stimulation based on input from the patient, wherein the patient changes one or more of the site, intensity or frequency of the TMS stimulation based on the patient's experience of the applied TMS stimulation; and applying Transcranial Magnetic Stimulation to the patient at the new site, intensity or frequency of TMS stimulation.

The method may also include the step of providing a stimulus to prompt a patient experience that is modified during the TMS procedure. Stimulus may be a stimulus that triggers, exacerbates or mimics the disorder, disease or behavior being treated. For example, the trigger may be an image of food when treating obesity/overeating, or a representation (sight/smell) of a drug or alcohol when treating addiction. Thus, the stimulus may comprise a visual stimulus, tactile stimulus, etc.

The step of changing one or more of the site, intensity or the frequency of the TMS stimulation may comprise allowing the patient to manipulate a handheld control to alter one or more of the site, intensity or frequency of the TMS stimulation. For example, the patient may move a joystick, toggle, dial, or other control during treatment. In some variations, the amount of control exerted by the patient during treatment may be limited. As mentioned, it may be limited or gated by unconscious patient feedback or input (e.g., heart rate, etc.). In some variations, the patient control may be limited to control within a range of values. For example, the patient may alter the site, intensity or frequency of the TMS stimulation only within a predetermined range for each of the site, intensity or frequency.

The step of changing one or more of the site, intensity or the frequency of the TMS stimulation may be performed while applying Transcranial Magnetic Stimulation to the patient. In some variations the patient control is exerted between ‘rounds’ of TMS stimulation.

Also described herein are patient-configurable Transcranial Magnetic Stimulation (TMS) methods that allows a patient to dynamically modify the TMS while a TMS procedure is being performed, the method comprising: positioning a plurality of TMS electromagnets to apply electromagnetic energy to a deep brain target site; applying TMS to the target site at a magnetic field intensity and a stimulation frequency; enabling the patient to change one or more of the position of the TMS electromagnet, the intensity of the TMS stimulation, or the frequency of the TMS stimulation based the patient's experience of the applied TMS stimulation; and applying Transcranial Magnetic Stimulation to the patient at the changed position of the TMS electromagnet, intensity of the TMS stimulation, or frequency of TMS stimulation.

As mentioned above, the method may also include providing a stimulus to prompt a patient experience that is modified during the TMS procedure. The stimulus comprises a visual stimulus, a tactile stimulus, a smell, a sound, etc.

As mentioned, the step of enabling the patient to change one or more of the position of the TMS electromagnet, the intensity of the TMS stimulation, or the frequency of the TMS stimulation may include allowing the patient to manipulate a handheld control.

Also described herein are systems for applying Transcranial Magnetic Stimulation (TMS), the systems comprising: at least one TMS electromagnet configured to apply TMS to a site in a patient's brain; a controller configured to control the TMS electromagnet to apply TMS to the site in a patient's brain at a magnetic field intensity and a frequency of stimulation; and a patient feedback input connected to the controller, configured to allow the patient to adjust one or more of the site of application of the TMS in the patient's brain, the magnetic field intensity of the applied TMS, or the frequency of the TMS stimulation during a TMS procedure on the patient.

In any of these systems, a plurality of TMS electromagnets configured to be positioned to apply TMS to a site in a patient's brain at a magnetic field intensity and a frequency of stimulation may be used.

The controller may be configured to coordinate the stimulation applied by a plurality of TMS electromagnets to apply TMS to a deep brain target. The patient feedback input may comprise a joystick, a mouse, a touch screen, a motion sensor, etc. As mentioned, the controller may be configured to limit the adjustment of the site of application of the TMS in the patient's brain, the magnetic field intensity of the applied TMS, and the frequency of the TMS stimulation by the patient feedback input so that these parameters remain within a predetermined range of values.

Also described herein are systems for applying Transcranial Magnetic Stimulation (TMS) including a plurality of TMS electromagnets configured to apply TMS to a deep brain target site in a patient's brain; a controller configured to control the plurality of TMS electromagnets to apply TMS to the target site in the patient's brain at a magnetic field intensity and a frequency of stimulation; and at least one patient feedback input configured to allow the patient to adjust one or more of the site of application of the TMS in the patient's brain, the magnetic field intensity of the applied TMS, or the frequency of the TMS stimulation during a TMS procedure on the patient.

### BRIEF DESCRIPTION OF THE DRAWINGS

FIG. 1 shows a circuit diagram of a full-wave rectifier circuit applied to a two-coil array.

FIG. 2 shows a circuit diagram of a half-wave rectifier circuit applied to a two-coil array.

FIG. 3 shows a full-wave bridge rectifier powering one double coil from an array. Note the intentional absence of a smoothing capacitor.

FIG. 4A illustrates the approximate waveforms of the electrical current input and outputs, respectively associated with the circuit shown in FIG. 1

FIG. 4B illustrates the approximate waveforms of the electrical current input and outputs, respectively associated with the circuit shown in FIG. 2.

FIG. 4C illustrates the approximate waveforms of the electrical current input and output, respectively associated with the circuit shown in FIG. 2.

FIG. 5A illustrates the placement of coils next to one another so as provide a similar orientation.

FIG. 5B illustrates the placement of coils on opposing sides of a patient's head so as to provide roughly opposite orientation.

FIG. 6 is a magnetic field profile of a primary TMS electromagnet alone, prior to addition of attractor magnet.

FIG. 7 is a magnetic field profile with attractor magnet in place.

FIG. 8A shows a figure-eight, double-coil electromagnet and magnetic-field profile.

FIG. 8B shows one configuration of figure-eight, double-coil TMS electromagnet, a high-permeability magnetic concentrator, and a target.

FIG. 8C illustrates a narrowed magnetic field profile for a figure-eight double-coil primary magnet in the presence of a high-permeability magnetic concentrator.

FIG. 9A shows three figure-eight, double-coil electromagnets and associated magnetic-field profiles and placement of target.

FIG. 9B illustrates the configuration of FIG. 9A showing narrowing of magnetic-field profiles and impact on target due to a high-permeability magnetic concentrator.

FIG. 10A is a diagram of a patient's mouth including one variation of a high-permeability magnetic concentrator that is not tailored to the shape of the patient's hard palette.

FIG. 10B is a diagram of a patient's mouth including a variation of a high-permeability magnetic concentrator that is tailored to the shape of the hard palette.

FIG. 11A shows a figure-eight, double-coil electromagnet and magnetic-field profile.

FIG. 11B illustrates one configuration of figure-eight, double-coil TMS electromagnet, a high-permeability magnetic concentrator, and a target.

FIG. 11C illustrates the configuration of FIG. 11B during stimulation, showing a narrowing of the magnetic-field profile and the impact on the target due to presence of the attractor magnet and two high-permeability concentrators.

FIG. 12A shows three figure-eight, double-coil TMS electromagnets and associated magnetic-field profiles and placement of target.

FIG. 12B shows the configuration of FIG. 12A with the addition of an attractor magnet and two high-permeability concentrators, illustrating a focusing of the magnetic-field profiles.

FIG. 13A shows one variation of an array of three double coil electromagnets supported on a three-sided frame, in which a coordinate (x, y, z) system has been overlaid.

FIG. 13B illustrates the frame shown in FIG. 13A moving in pitch.

FIG. 13C illustrates the frame shown in FIG. 13A moving in roll.

FIG. 13D illustrates the frame shown in FIG. 13A moving in yaw.

FIG. 14 shows an array of three figure-eight, double-coil electromagnets supported on a three-sided frame.

FIG. 15 illustrates another array of electromagnets configured so that entire array may move in roll, pitch, and yaw.

FIG. 16 Illustrates one variation of single TMS electromagnet pair configured to move in pitch, roll and yaw.

FIG. 17 shows one variation of a TMS actuator module.

FIG. 18 illustrates one variation of a TMS method including a method for adjusting the position of a TMS electromagnet at different positions relative to a patient's head using a circular gantry.

FIG. 19 illustrates a gantry with multiple electromagnets attached to plates that run on a track around the gantry in end-to-end fashion.

FIG. 20 illustrates another variation of an oval gantry having two electromagnets.

FIG. 21 schematically illustrates a TMS system as described herein.

FIG. 22 illustrates one TMS method as described herein.

FIG. 23 illustrates a customizable TMS gantry.

FIG. 24 is a flow chart illustrating one variations of the method for intra-session control of TMS.

FIG. 25 shows one variation of a system for patient-configurable TMS.

FIG. 26 is a table of therapies, deep-brain TMS targets and exemplary acute patient feedback.

### DETAILED DESCRIPTION OF THE INVENTION

### Part I: Monophasic Multi-Coil Arrays for Transcranial Magnetic Stimulation

Described below is the use of one or more (e.g., an array) of TMS electromagnets to stimulate the brain treatment of hypertension.

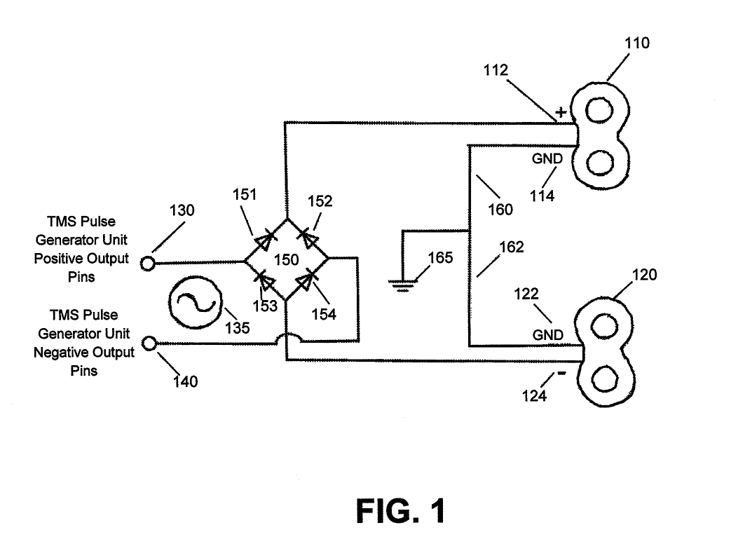

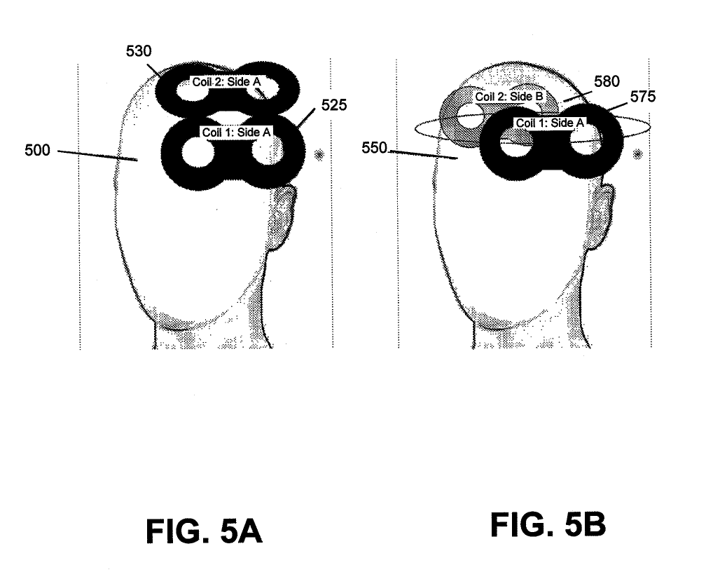

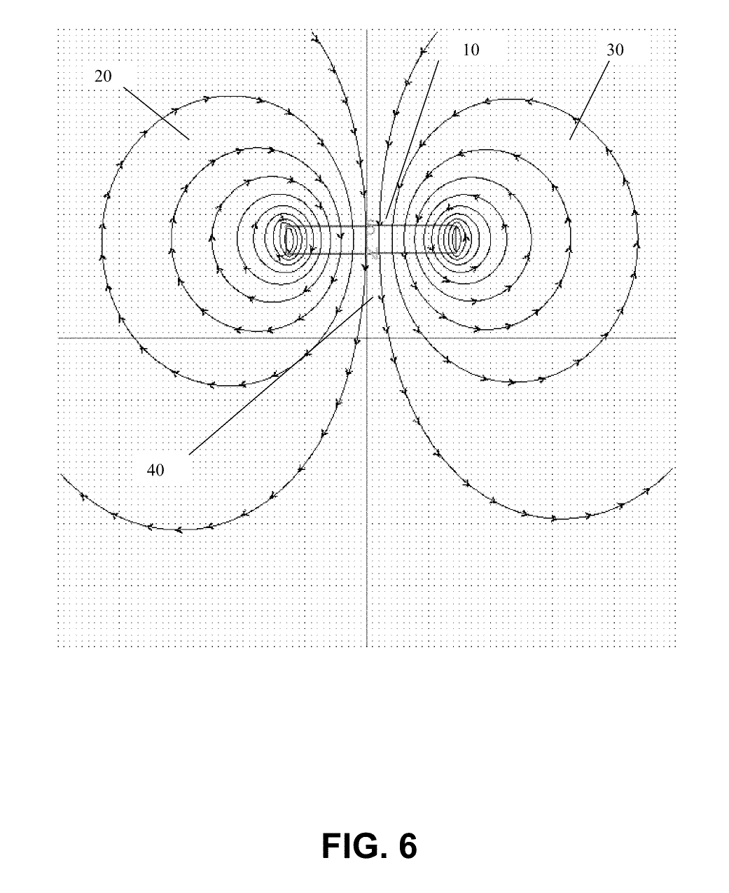

FIG. 1 shows a circuit diagram of a full-wave rectifier circuit applied to a two-coil array. In this particular embodiment, the two coils in the array, coil 110 and coil 120 are double coils, for example 70 mm double coil manufactured by Magstim Ltd. (Wales, UK). In such a double coil, two separate concentric windings are wrapped in opposite directions with a crossover between the two portions, placed such that the positive and the negative going leads to the two coil portions run electrical current in the same direction where the two portions are adjacent to one another, creating the greatest magnetic field induction under the center. Positive electrical pole 130 and negative electrical pole 140 have a pulsatile polyphasic alternating current 135 between them, and represent the black and red output wire pins on a standard repetitive transcranial magnetic stimulation device. The positive going current from pole 130 and negative going current from pole 140 enter bridge rectifier 150, which is composed of diodes 151, 152, 153 and 154. Positive portions of the waveform are then sent to positive pole 112 of TMS coil 110, while negative-going portions of the negative pole 124 of TMS coil 120. Ground pole 122 of TMS coil 120 and ground pole 114 of TMS coil 110 both go to ground 165 by leads 162, and 160, respectively. The output of this circuit will be illustrated in FIG. 4 A, and positioning of these coils will be discussed with respect to FIGS. 5 A and 5B. The electrical pulses through each of the coils is thereby monophasic, and the induced magnetic field, is at least substantially monophasic.

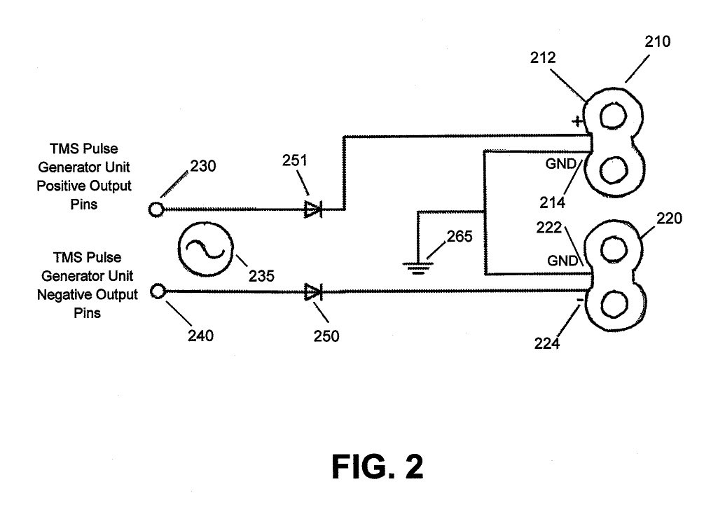

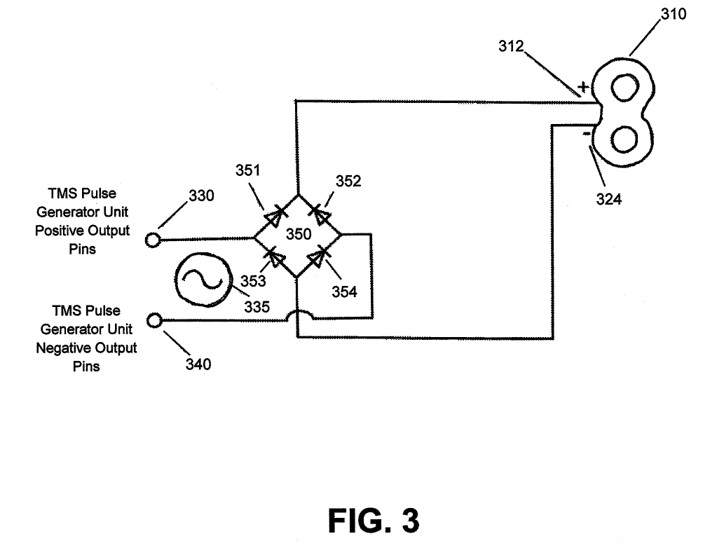

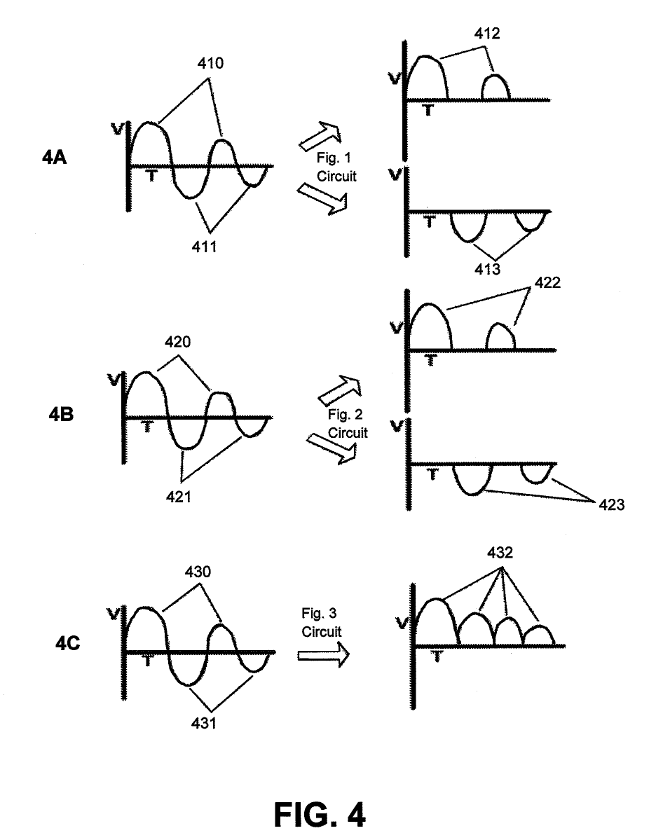

FIG. 2 shows a circuit diagram of a (half-wave) rectifier diodes applied to a two-coil array. Positive electrical pole 230 and negative electrical pole 240 have a pulsatile polyphasic alternating current 235 between them, and represent the black and red output wire pins on a standard repetitive transcranial magnetic stimulation device. The positive going current from pole 230 enters diode 251 and is passed to positive pole 212 of TMS coil 210. Meanwhile, the negative-going current from pole 240 enters diode 250 and is passed to negative pole 224 of TMS coil 220. Ground pole 222 of TMS coil 220 and ground pole 214 of TMS coil 210 both go to ground 265. The output of this circuit will be illustrated in FIG. 4B, and positioning of these coils will be discussed with respect to FIGS. 5A and 5B. The electrical pulses through each of the coils is thereby monophasic, and the induced magnetic field, is at least substantially monophasic.

FIG. 3 illustrates the use of a full wave bridge rectifier circuit as applied to a single coil. Positive electrical pole 330 and negative electrical pole 340 have a pulsatile polyphasic alternating current 335 between them, and represent the black and red output wire pins on a standard repetitive transcranial magnetic stimulation device. The positive going current from pole 330 and negative going current from pole 340 enter bridge rectifier 350, which is composed of diodes 351, 352, 353 and 354. Positive portions the waveform are then sent to positive pole 312 of TMS coil 310, while negative-going portions of the negative pole 324 of TMS coil 310. The output of this circuit will be illustrated in FIG. 4C. The electrical pulses through the coils is thereby monophasic, and the induced magnetic field, is at least substantially monophasic.

FIG. 4A illustrates the input and outputs of the circuit shown in FIG. 1. Positive going pulses 410 and negative-going pulses 411 are input into the FIG. 1 circuit, and emerge as positive-going pulses 412 on one TMS coils, and negative-going pulses 413 on the other TMS coil. When using this configuration, the two coils may be arranged on opposite sides of the head so as to maximize summation and minimize energy cannibalization, provided that the coils are properly flipped so as to summate rather than cancel, as will be described with respect to FIGS. 5A and 5B.

FIG. 4B illustrates the input and outputs of the circuit shown in FIG. 2. Positive-going pulses 420 and negative-going pulses 421 are input into the FIG. 1 circuit, and emerge as positive-going pulses 422 on one TMS coils, and negative-going pulses 423 on the other TMS coil. When using this configuration, the two coils may also be arranged on opposite sides of the head so as to maximize summation and minimize energy cannibalization, provided that the coils are properly flipped so as to summate rather than cancel, as will be described with respect to FIGS. 5A and 5B.

FIG. 4C illustrates the input and output of the circuit shown in FIG. 3. Positive-going pulses 430 and negative-going pulses 431 are input into the FIG. 3 circuit, and emerge as rectified positive-going pulses 432 the TMS coil. In typical rectifier circuits, a capacitor is generally used to smooth the output. However, in the present invention, no capacitor is used, as unevenness, with its associated DB/DT is a desirable quality for inducing neuronal depolarization.

FIG. 5 describes two forms of spatial relationship that two or more coils in an array can have in boost the summation of their fields. FIG. 5A shows two TMS coils; coil 530 and coil 525 placed alongside one another over head 500. While their positions are obviously not identical due to space that each takes up on the scalp surface, it will be appreciated that their trajectories are substantially similar. In practice this means that a significant percentage of their magnetic field output of the same phase will sum rather than cancel. FIG. 5B shows two coils; coil 575 and coil 580 facing each other from opposite sides of the patients' head 550. In practice, this position will generally cause pulses from the two coils to summate only if they are in opposite phase.

### Discussion of Part I

Using the means provided herein, biphasic electrical pulse source are divided such that the current passing through each of the coils is monophasic, and the induced magnetic field, is at least substantially monophasic. Because magnetic field is induced as a function of change in electrical current per unit time, even a perfectly monophasic electrical pulse does not create a perfectly monophasic magnetic field pulse: the pulse of electrical current cannot continue to rise indefinitely (in practice the current pulses are typically of approximately 0.1 ms in duration), and then falls to baseline. This means that there will be some reversal of induced magnetic field direction. However, in accordance with the means provided, this opposite-phase component will be of substantially less magnitude than the principal component of the magnetic field pulse.

Summation from two sources from standpoint of physics (as opposed to temporal and spatial forms of physiological summation) can be summarized as follows:

Matched phase and opposite direction yields cancellation.

Opposite phase and similar direction yields cancellation.

Matched phase and similar direction yields summation.

Opposite phase and opposite direction yields summation.

In practice under general conditions, however, side-by-side coils of same polarity and phase may either enhance or diminish the neuronal response, as the sharp DB/DT at the margin between these coils may produce a potent depolarizing effect. Likewise coils on the opposite side of the head of opposite polarity are subject to the same uncertainty. Therefore controllability of this phenomenon depends upon precise control of magnetic field direction and as well as precise controls of the coils with respect to specific anatomical geometry.

Note that phase can be changed electronically, as described herein, reversing polarity of the coil, or by simply flipping the coil face with respect to the target. In accordance with the present invention, coil polarity and direction of aim are planned in order to maximize the extent to which vectors that are desired are summated, and vectors which are not desired are cancelled.

The ability of those two coils to act synergistically to depolarize neurons, rather than to diminish each other's effects, however, depends upon depends upon coordinating the coil polarity, the pulse phase and the pulse timing, as well as the power of the sources. For example, two coils on the opposite sides of a patient's head, located 180 degrees apart, but with like coil faces (same polarity with respect to the head) may serve to diminish the stimulating effect in the area between the coils. By contrast, two side-by side magnetic coils oriented in essentially the same polarity, and pulsed simultaneously from identical sources, will summate to produce additive effects. Likewise, two coils on the opposite sides of a patient's head, located 180 degrees apart, but with one coil face flipped to provide opposite polarity, will also result in an additive effect in the space between the coils.

### REFERENCES

Dantec Magnetic Stimulation Product Information on MagPro X100 with MagOption. http://www.danica.nl/neuro/neuro-magnetische-stimulatoren.htm.
Magnetic Stimulation in Clinical Neurophysiology. Second Edition. Hallet M, Chokroverty S, Ed. Elsevier Inc., Philadelphia, Pa., 2005. Chapter by Ruohonen, et al.
Transcranial Magnetic Stimulation: A Neurochronometrics of Mind. Walsh V, Pascual-Leone A. MIT Press. Cambridge, Mass. 2003.
Davey K., Riehl M., “Deigning Designing Transcranial Magnetic Stimulation Systems,” IEEE Transactions on Magnetics. Vol. 41, No. 3, March 2005. 1142-1148.
Barker, A. T., “An Introduction to the Basic Principles of Magnetic Nerve Stimulation,” Journal of Clinical Neurophysiology. Vol. 8, No. 1, 1991, 26-37.
“Robotic Device for Stereotactic Transcranial Magnetic Stimulation.” M. B. Schneider and D. J. Mishelevich, U.S. patent application Ser. No. 10/821,807.
“Trajectory-Based Transcranial Magnetic Stimulation,” D. J. Mishelevich and M. B. Schneider, U.S. patent application Ser. No. 11/429,504.

### Summary of Part I

Efficient use of multi-coil arrays for magnetic nerve stimulation depends upon coordinating the coil polarity, the pulse phase and the pulse timing. Monophasic magnetic nerve stimulators produce more precise and predictable results in the stimulation of nerves than biphasic and polyphasic machines, but are less electrically efficient, and consequently limited in terms of pulse train speed. The present invention concerns the coordination of pulse polarity, phase, timing, and strength between multiple magnetic stimulation coils. The goal is to optimize the manner in which multiple coils may be used synergistically to control the activity of underlying neural tissue.

### Part II: Focusing Magnetic Fields with Attractor Magnets and Concentrator Devices

Part II describes devices and methods that relate generally to the focusing of magnetic fields generated by electromagnets used for Transcranial Magnetic Stimulation.

Described herein are Transcranial Magnetic Stimulation (TMS) systems, methods and devices that include one or more magnetic field (or flux) modifying elements, including magnetic concentrators and/or attractor magnets. These magnetic field modifying elements may be configured to focus the electromagnetic field applied by one or more primary TMS electromagnetic coils.

As described briefly above, an attractor magnet is typically a secondary magnet that is configured to produce a magnetic field that interacts with and modifies the electromagnetic field produced by the primary TMS electromagnet(s). The attractor magnet is typically positioned opposite of (or otherwise separate and across from) the primary TMS electromagnet. Thus, in some variations, the attractor magnet is opposite in polarity in a phase-complementary manner at any given time with the primary electromagnet magnet.

Thus, described herein are systems for stimulating a subject's neuronal tissue, which may include a primary electromagnet configured to apply Transcranial Magnetic Stimulation to the subject, and an attractor magnet, wherein the attractor magnet is configured to be positioned opposite the primary electromagnet and to apply an electromagnetic field that is opposite in polarity in a phase-complementary manner at any given time with the primary electromagnet magnet.

In general, these systems may also include one or more controllers. For example, the system may include a controller configured to coordinate activation of the primary electromagnet and the attractor magnet so that the TMS applied by the primary electromagnet is focused by the attractor magnet on the neuronal target. The controller may be part of an overall TMS system controller, or it may be a separate controller. The controller may include controls for actively positioning the primary electromagnet and/or the attractor magnet. The controller may sense or receive input on the position of the primary electromagnet and/or the attractor magnet and the target, and may control the energy applied to activate one or both the primary electromagnet and the attractor magnet so as to focus the applied TMS on the target in a desired manner. Thus, the controller may guide the system in applying the TMS to deeper tissue region or applying TMS in a more uniform and focused manner than TMS without the use of an attractor magnet. The controller may also help assure that the electromagnet field of the attractor magnet is opposite in polarity in a phase-complementary manner at any given time with the primary electromagnet magnet.

Any of the systems described herein may also include a second (or additional) primary TMS electromagnets. Additional attractor magnets may also be used.

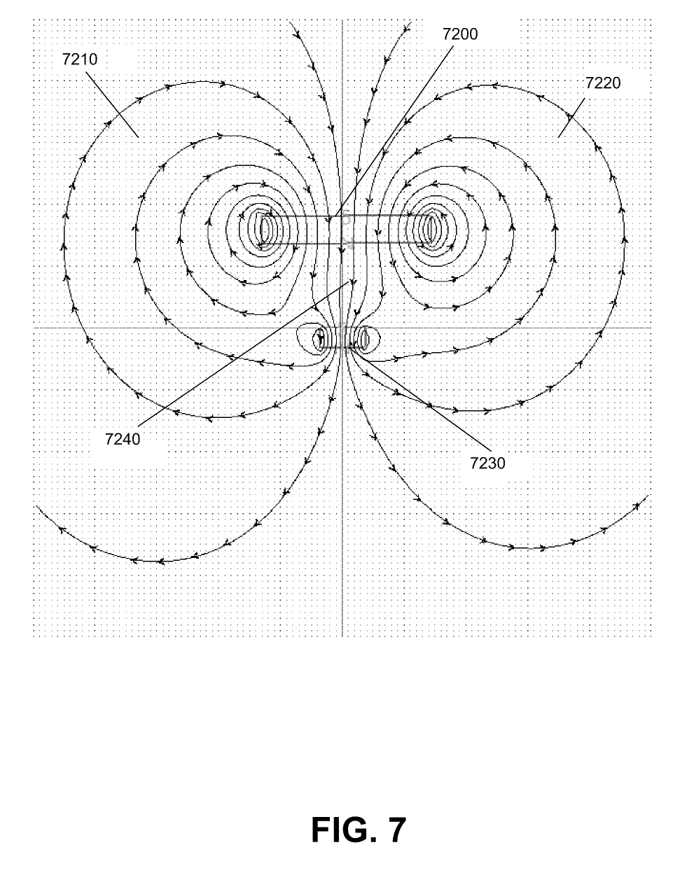

FIGS. 6 and 7 both illustrate a primary of primary magnet 10 (FIG. 6), 7200 (FIG. 7). In FIG. 6, the primary TMS magnet 10 has magnetic field lobes 20 and 30, and a magnetic field flux density measured at point 40 of 1.54×10⁻⁴ units. In FIG. 7, the primary magnet 7200 has lobes 7210 and 7220, and also illustrates an attractor magnet 7230. In FIG. 7, the magnetic field flux density measured at a location 240, which is equivalent to position 40 of FIG. 6, the measured field flux is 2.79×10⁻⁴ units, with both the primary magnet and the attractor magnet in place. In comparison, the field strength measured with just the attractor magnet alone is 1.89×10⁻⁴ units. This is because the attractor magnetic is of opposite and phase-complimentary (temporal and/or spatial) orientation with respect to primary magnet 7200. This configuration and relationship operates similarly even if the primary and attractor magnets do not directly face each other.

Any appropriate magnet may be used as an attractor magnet as described herein. Thus, the size, shape, and power of the attractor magnet may be configured to best modify the electromagnetic field emitted by the primary magnet(s). For example, coil or other shapes may be used. In operation, the attractor magnet may be positioned either opposite the primary magnet(s), or at an angle to the primary magnets. The system may be configured so that the relationship (e.g., angle and/or distance) between the attractor magnet and the primary magnet is consistent or stereotyped. In some variations the attractor magnet is configured to be applied externally to the patient (e.g., around the patients head), or within the patient (e.g., within the mouth, ear, nasal regions, etc.).

Alternatively or in addition to the attractor magnets described herein, the system may also include one or more magnetic concentrators. A described briefly above, a magnetic concentrator is typically a device including a material having a relatively high magnetic permeability. Exemplary materials include nickel-iron alloys such as permalloy, and “mu-metal”. Mu-metal is a nickel-iron alloy (75% nickel, 15% iron, plus copper and molybdenum) that has very high magnetic permeability.

A magnetic concentrator may be shaped to direct the magnetic flux lines of the primary magnet in a desired fashion. In some variations, the magnetic concentrator may direct the flux lines to focus the magnetic field applied by the primary electromagnet. Similarly, a magnetic concentrator may be applied to divert the magnetic field applied by the primary TMS electromagnet from non-target tissue regions. This may help prevent unwanted stimulation of non-target neuronal regions, for example.

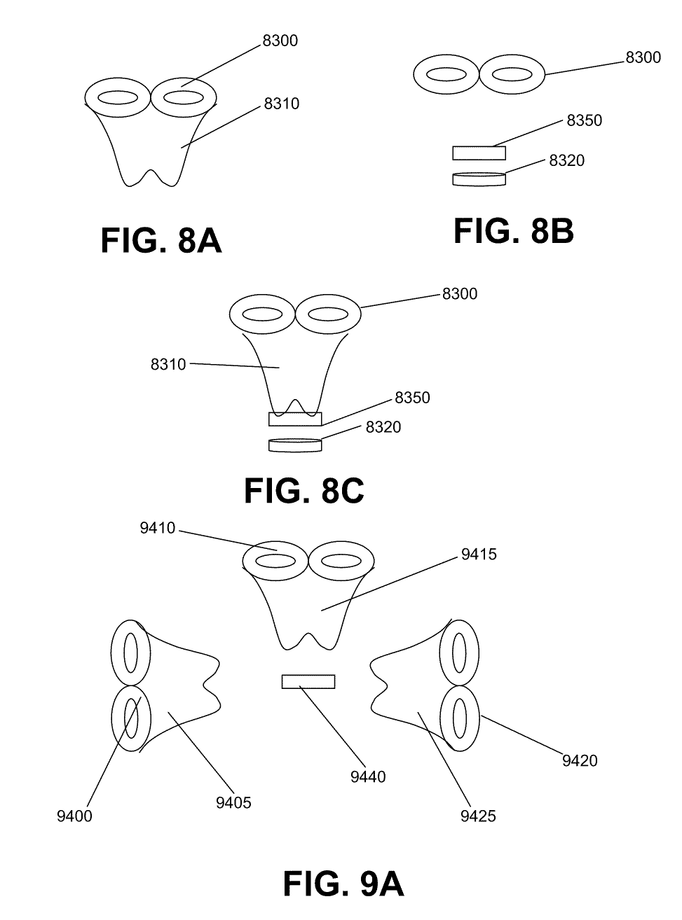

FIG. 8A shows a simplified schematic view of an electromagnetic field generated by a figure-eight shaped primary TMS electromagnet. The width of the generated magnetic field 8310 is illustrated. Such figure-eight double coils are well known, for instance the 70 mm double-coil configuration from Magstim (e.g., Model 9925, Magstim Ltd., Wales, UK). The electromagnets can be powered by commercially available power sources such as the “Magstim Rapid²” (Magstim Ltd., Wales, UK) that provide electrical currents for pulsed magnetic fields. A controller may control the power source.

FIGS. 8B and 8C illustrate the effect of one variation of a magnetic concentrator on the primary electromagnet shown in FIG. 8A. In FIG. 8B, the same electromagnet 8300 shown in FIG. 8A is positioned opposite a target region 8350 and a magnetic concentrator 8320. The magnetic concentrator includes a high-permeability concentrator region 8320 that is placed close to the target 8350 and can augment the field strength due from the primary electromagnet 8300 at the target of interest 8350. FIG. 8C illustrates the resulting narrower magnetic field 8310 due to the concentration of the field generated by the primary electromagnet 8300 and the magnetic concentrator 8320. Thus, the magnetic concentrator results in a more intense magnetic field at target 8350 than would otherwise be present.

The concentrator's high-permeability concentration region can be a small region that may help focus the magnetic field at the given location. By reducing the dimension of the concentrator that is parallel to the primary magnet, while concurrently increasing the perpendicular dimension, the size and mass of the concentrator can be maintained, thereby preserving its magnetic properties, and increasing the focus from the concentrator. The high-permeability concentrator region of the magnetic concentrator acts to concentrate the applied magnetic field and thus augments the magnetic field at that target location compared to what it would have been if the high-permeability region were not present. The effect applies whether or not the electromagnet and high-permeability regions directly face each other or not. In FIG. 8A-8C, the high permeability regions shown are in fixed configurations with the primary electromagnet. In an alternative embodiment, the primary electromagnet can move (for example, as described in U.S. patent application Ser. No. 10/821,807). The fixed-configuration high-permeability regions may be selected from a set of available alternatives.

As mentioned, the high-permeability magnetic region can be made of one or more materials, including MuShield (MuShield Company, Londonderry, N.H.), NETIC® and CO-NETIC® alloys from Magnetic Shield Corporation (Bensenville, Ill.), and AD-MU alloys from Ad-Vance Magnetics, Inc. (Rochester, Ind.).

One or more source electromagnets (primary TMS electromagnets) and/or one or more targets may be used with one or more magnetic concentrators. For example, FIG. 9A shows a variation in which three figure-eight coil pairs 9400, 9410, and 9420 generating magnetic-field profiles (9405, 9415, and 9425, respectively) are aimed towards a target 9440. In FIG. 9B, the same configuration is used, but also including a magnetic concentrator placed reasonably near the target. In comparison to the magnetic field profiles shown in FIG. 9B, the same electromagnets 9400, 9410, and 9420 now generate narrower magnetic field profiles 9407, 9417, and 9420 than the magnetic field profiles 9405, 9415, and 9425 shown in FIG. 9A, because of the presence of the magnetic concentrator 9420. The focused magnetic fields 9407, 9417, and 9427 evoked with the concentrator have a greater magnetic-field impact on target 9440 in FIG. 9B than would otherwise occur with unfocused magnetic fields. Thus, the focused magnetic fields applied may penetrate deeper, and over a smaller (or larger) target area than without a magnetic concentrator, depending on the configuration of the magnetic concentrator relative to the primary magnet(s).

The magnetic concentrator may be configured as a re-useable or as a disposable device or system element. For example, the magnetic concentrator may be configured as an adhesive patch that is applied to the subject's head, or internally to the subject's head. In some variations the magnetic concentrator is configured as an implant that is temporarily or chronically implanted in the subject. The magnetic concentrator including the high-permeability region may be configured as a disposable or re-useable device whose shape is customized to each individual patient. Such concentration devices may be disposables in the sense that they are used for only one patient, but need not necessarily be disposed of between individual sessions with the same patient. Additionally these devices may become magnetically saturated over a period of use, and therefore require replacement with a fresh, unsaturated device.

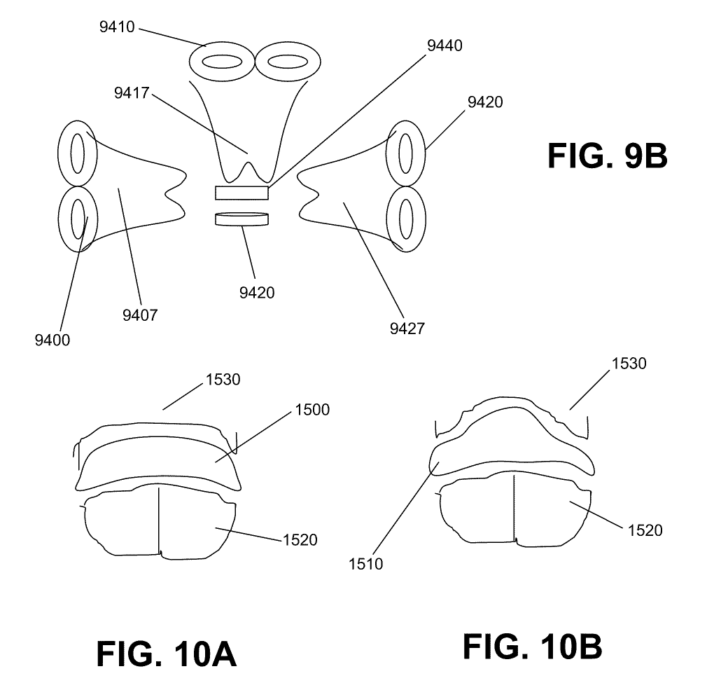

FIGS. 10A and 10B show examples of magnetic concentrator devices configured to be held in a patient's mouth during TMS. For example, in FIG. 10A, component 1500 is positioned vertically between the tongue 1520 and the palette 1530 of the subject's mouth. In FIG. 10A, the shape profile of high-permeability concentrator component 1500 is lower to approximately match the shape of the underside of palette 1530. In FIG. 10B, the magnetic concentrator component 1510 extends noticeably higher to accommodate a high palette 1530, because the magnetic concentrator has been custom formed to fit the patient's anatomy in this region. The target (not shown) in this example, is typically located superior to the palette 1530. Other shapes for high-permeability magnetic concentrator components (disposable or re-usable) may fit other physical cavities, such as the nasal cavity, sinus cavities, the oral or nasal pharynx, or the external ear canal. The regions can incorporate passages for air or fluid to maintain physiological function including diagnostic and therapeutic elements. In addition, the shape may be configured to fit outside regions of the body, such as the head, neck or face. Different shapes in different cavities can be used simultaneously. For example nasal-cavity inserts can be used in conjunction with buccal component inserts. A variety of shapes can be employed, although some will be more effective at focusing than others. The third dimension of a given concentrator may be of the same or an alternate shape. The high-permeability region of the concentrator need not be symmetric. In some variation a single magnetic concentrator includes multiple sub-regions comprised of high magnetic permeability material, which may allow further shaping or refining of the applied electromagnetic field from the primary electromagnet.

A magnetic concentrator (including the high-permeability region or regions of the magnetic concentrator) may be molded to fit the available space. In some variations, the magnetic concentrator may be compress-able, expandable, or otherwise anchorable, and may be configured for insertion, for example, it may include appendages or embodiments allowing for expansion and contraction (e.g., an umbrella-like). The component may be held in place by conformation with a cavity or with a suitable fixture. Examples of embodiments include filling up the rest of the space with resilient or non-resilient foam or placing a spring (which may be non-ferromagnetic) against an opposing surface.

A magnetic concentrator may also be used to control the flux pathway of a primary electromagnet and thereby protect non-target tissues. For example, in some variations, the system may include one or more magnetic concentrators with high-permeability regions that are placed on the same side of a primary electromagnet relative to a target, or are placed adjacent to the target. These positions may allow the magnetic concentrator to guide the flux pathway away from non-target regions, particularly regions that it would be desirable to avoid over-stimulation of the target. Thus, a magnetic concentrator may be placed at any useful position or orientation relative to the source electromagnet(s) and the target(s).

As mentioned the magnetic field modifying elements described herein need not only be external, but may be applied internally as well. For example, external implementations may be placed under the chin or on the outside of the cheeks of the face or on the temples. Any suitable shape is appropriate. For example, a magnetic concentrator may include a high-permeability region may be configured as a horseshoe-shape which may be more effective than the rounded shape of a sphere. Components need not always match the shape of a cavity into which they are applied, such as the oral cavity, nasal cavity, etc., or the surface to which they are applied. As mentioned, a magnetic concentrator may be placed implanted with the patient, e.g., under anesthesia. For example this approach can be used in connection with a component placed in the nasal pharynx. In one embodiment, the high-permeability region can be constructed in liquid or paste form such that it can be injected into a cavity with a small opening such as a sinus cavity, the procedure performed, and then the high-permeability liquid form removed. Examples of such materials are found in Xiao et al., 2005 and Yoshida et al., 2005 (U.S. Pat. No. 6,792,097). In another embodiment, the magnetic concentrator including a high-permeability component could be inserted into the cranium through an open-craniotomy procedure. Such an implant could be left in permanently or removed later. In some cases, the implant would be placed during open surgery not done for the sake of the implant alone but done for another purpose such as therapy for intra-cranial bleeding. The high-permeability component could then be used for therapy to restore function temporarily lost due to the lesion such as intra-cranial bleeding.

External magnetic concentrators can draw magnetic field to an external location or through a volume at an intermediate position between the magnetic source and the magnetic region. In certain circumstances the high-permeability component may be used to concentrate magnetic field locally to prevent penetration of the field to sensitive underlying structures that need to be protected (say as a source electromagnet is rotating around the head). A typical embodiment may be an external shield or set of shields at the side or sides of the head or face. This use of shielding includes drawing away all or a portion of the magnetic field from superficial or other areas that, if over-stimulated, might generate seizures.

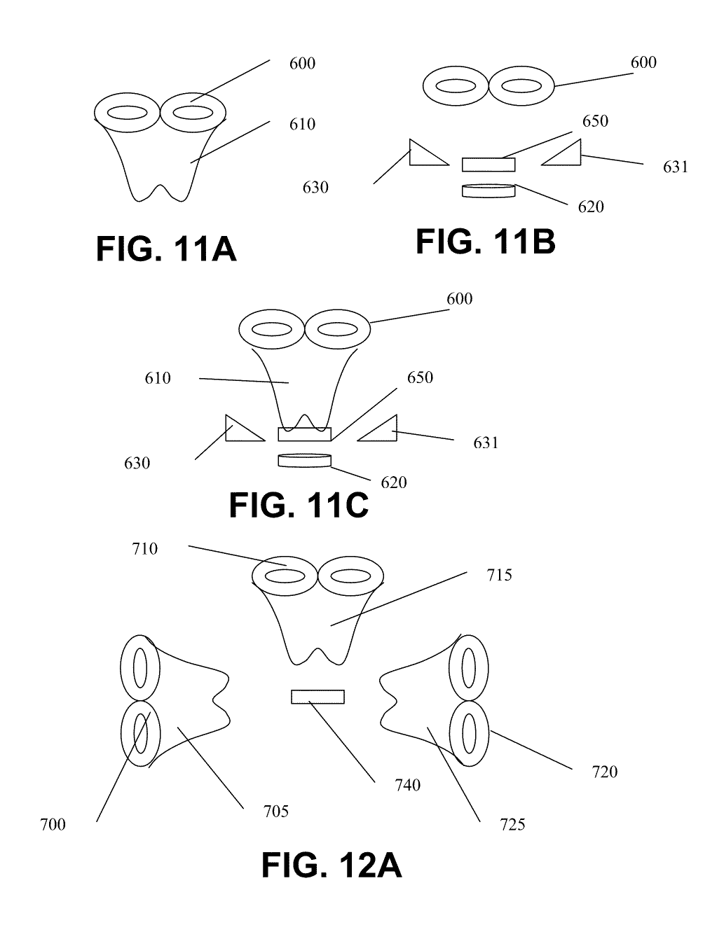

FIG. 11A illustrates another example of a magnetic field 610 resulting from a figure-eight electromagnet 600. As mentioned above, such figure-eight double coils are well known. This double-coil electromagnet is used as only one example of a primary electromagnet; any appropriate primary TMS electromagnet may be used with the systems and methods described herein. The electromagnets can be powered by available power sources such as the Magstim Rapid (Magstim Ltd., Wales, UK) that provides for pulsed magnetic fields, and may be controlled by a controller, as described.

In some variation, a system may include both an attractor magnet and a magnetic concentrator. As described above, the attractor magnet may be any active or “source” electromagnet other than the primary source magnet, which is typically arranged at a position opposite the primary source magnet. The attractor magnet, owning to the opposite phase if its pulses relative to the primary source magnet, serves to pull magnetic field into the space interposed between the primary magnet and the attractor magnet. For example, FIG. 11B shows one variation of a system in which an attractor magnet 620 and two magnetic concentrators 630, 631 are used. In this configuration, which uses the same electromagnet 600 in FIG. 11A, for which an attractor magnet 620 and high permeability concentrator regions 630 and 631 are placed close to a target 650, can augment the field strength due to electromagnet 600 at the target of interest 650. FIG. 11C shows the narrower magnetic field 610 resulting from the focusing or concentration of the field generated by electromagnet 600 due to attractor magnet 620 and magnetic concentrators 630 and 631. Thus, a more intense magnetic field occurs at target 650 than there would be otherwise. In other embodiments attractor magnets and concentrator devices can be used to widen the magnetic-field profile of the primary magnet.

In general, the attractor magnets and magnetic concentrators described herein concentrate the applied magnetic field and thus augment the magnetic field at a target location compared to what it would have been if these were not present. The effect applies whether or not the primary electromagnet and one or more attractor magnets and high-permeability regions directly face each other or not. As previously mentioned, although the attractor magnets and high-permeability regions shown in these embodiments are in fixed configuration relative to the primary electromagnet, the primary electromagnet may be mobile (for example as described U.S. patent application Ser. No. 10/821,807); in addition one or both of the magnetic concentrator(s) and/or attractor magnet(s) may also be mobile, and may move synchronously with the primary electromagnet. In other embodiments the attractor magnets, the concentrator devices or both can move, with or without movement of the primary source electromagnet.

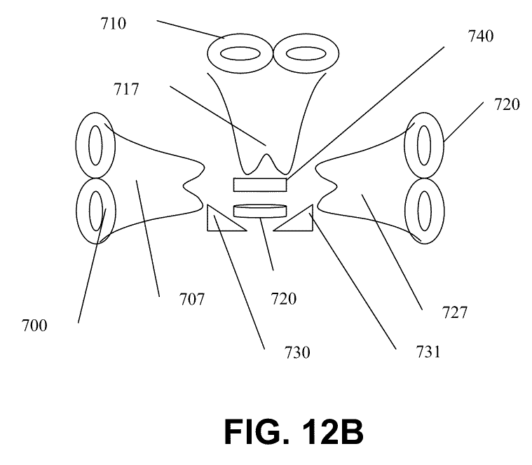

One or more source electromagnets may be used or form a part of the systems described herein, and one or more targets may be stimulated with TMS using these stems and methods. For example, FIG. 12A, illustrates three figure-eight coil pairs 700, 710, and 720 generating magnetic-field profiles 705, 715, and 725 respectively aimed towards target 740. FIG. 12B illustrates a system with the same three primary electromagnets, but also including a pair of magnetic concentrators and an attractor magnet. In comparison to the magnetic field profiles shown in FIG. 12A, the same electromagnets 700, 710, and 720 now generate narrower magnetic-field profiles 707, 717, and 727 than the profiles 705, 715, and 725 because of the presence of attractor magnet 720 and high-permeability concentrator regions 730 and 731. The combination of the magnetic fields 707, 717, and 727 has greater magnetic-field impact on target 740 in FIG. 12B than would otherwise occur with unfocused magnetic fields.

Thus, described herein are Transcranial Magnetic Stimulation systems for stimulating a subject's neuronal tissue that may include a primary electromagnet configured to apply Transcranial Magnetic Stimulation to the subject and a magnetic concentrator comprising a shaped region of high magnetic permeability, wherein the concentrator is configured to modify the Transcranial Magnetic Stimulation applied by the primary electromagnet. In some variations, the system also includes an attractor magnet, wherein the attractor magnet is configured to be positioned opposite the primary electromagnet and to apply an electromagnetic field that is opposite in polarity in a phase-complementary manner at any given time with the primary electromagnet magnet.

As mentioned above, any of these systems may also include a controller configured to coordinate the positions of the primary electromagnet and the magnetic concentrator relative to a neuronal target.

In operation, any of the devices and systems described herein may be used to apply TMS to a patient in need thereof. For example, TMS may be used to treat one or more disorders (e.g., depression, chronic pain, addiction, obesity, and obsessive compulsive disorder, or other psychological disorders) using any of the devices and systems, including the attractor magnets and magnetic concentrators, described.

In general, a subject may be treated by providing TMS stimulation after first positioning the primary electromagnet(s) and any additional magnetic field modifying elements, such as attractor magnets and/or magnetic concentrators. The step of positioning either or both the primary electromagnet(s) and the additional magnetic field modifying elements may be guided, e.g., by the controller, which may determine an optimal position based in part on target position. For example, a controller may detect or be told (e.g., by direct input) the selected target(s) and the position of the system components such as the primary electromagnet(s), magnetic concentrator(s), and attractor magnet(s). After positioning these components, the system may then determine the desired stimulation protocol to achieve targeted stimulation without undesirably stimulating (or over stimulating) non-target tissues.

For example, in variations in which the target selected is a deep-brain (e.g., non-cortical) target, it may be desirable to avoid stimulating neural tissue that is located superficially to the deep target. Thus, the controller (or other portion of the system) may determine the appropriate stimulation protocol to achieve stimulation at a desired frequency, rate and/or duration to activate or inhibit the target without stimulating (or overstimulation) non-target regions. In determining the stimulation protocol, the system may calculate the effect of any attractor magnet(s) and/or magnetic concentrator(s) including in the system. For example, the controller may receive input on the position and orientation, as well as the magnetic properties of the attractor magnet(s) and magnetic concentrator(s) (e.g., the field strength range of the attractor magnet(s) and the magnetic permeability of the magnetic concentrator(s)). This information may be used to determine the effect and optimize the treatment protocol.

For example, described herein are Transcranial Magnetic Stimulation methods for stimulating a neuronal target tissue that include the steps of: selecting the neuronal target; positioning a primary electromagnet to apply electromagnetic energy to the target; positioning an attractor magnet opposite the primary electromagnet; emitting an electromagnetic field from the primary electromagnet; and emitting an electromagnetic field from the attractor magnet that is opposite in polarity in a phase-complementary manner at any given time with the electromagnetic field emitted from the primary electromagnet magnet, so that the magnetic field applied to the neuronal target from the primary electromagnet is focused by the electromagnet field from the attractor magnet. The method may also include the step of positioning a magnetic concentrator on or within the subject to enhance the electromagnetic energy applied to the target from the primary electromagnet. Alternatively (or additionally), the method may include the step of positioning a magnet concentrator on or within the subject to shield a region of the subject from the electromagnetic energy applied to the target.

In some variations, the method includes determining the energy applied to the primary electromagnet based on the positions of the target, primary electromagnet and attractor electromagnet.

Also described herein are Transcranial Magnetic Stimulation methods for stimulating a neuronal target tissue that include the steps of: selecting the neuronal target; positioning a primary electromagnet to apply electromagnetic energy to the target; positioning a magnetic concentrator on or within the subject to enhance the electromagnetic energy applied to the target from the primary electromagnet; and emitting an electromagnetic field from the primary electromagnet so that the emitted electromagnetic field is altered by the magnetic concentrator.

The step of positioning the magnetic concentrator may include positioning the concentrator within the subject's body (e.g., within the nose, mouth, ears, etc.). The magnetic concentrator may be a disposable magnetic concentrator, or a re-usable one. In some variations, the step of positioning the magnetic concentrator comprises applying the magnetic concentrator comprises applying the magnetic concentrator to the subject's head.

Also described herein are Transcranial Magnetic Stimulation methods for stimulating a neuronal target tissue that include: selecting the neuronal target; positioning a primary electromagnet to apply electromagnetic energy to the target; positioning a magnetic concentrator on or within the subject to shield a region of the subject from the electromagnetic energy applied to the target by the primary electromagnet; and emitting an electromagnetic field from the primary electromagnet so that the emitted electromagnetic field is altered by the magnetic concentrator.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, which is set forth in the following claims.

### REFERENCES

Avery, D. H., Holtzheimer III, P. E., Fawaz, W., Russo, Joan, Neumaier, J. and Dunner, D. L., Haynor, D. R., Claypoole, K. H., Wajdik, C. and P. Roy-Byrne, “A Controlled Study of Repetitive Transcranial Magnetic Stimulation in Medication-Resistant Major Depression,” Biological Psychiatry, 2005, 59:187-194.
Chris Hovey C, Houseman P and Jalinous R. The New Guide to Magnetic Stimulation. The Magstim Company Ltd Carmarthenshire, United Kingdom 2003.
D. J. Mishelevich and M. B. Schneider, U.S. patent application Ser. No. 11/429,504 “Trajectory-Based Deep-Brain Stereotactic Transcranial Magnetic Stimulation”.
Riehl; M. E. et al., U.S. Patent Application Publication No. 2006-0122454, “Reducing Discomfort Caused by Electrical Stimulation.”
M. B. Schneider and D. J. Mishelevich, U.S. patent application Ser. No. 10/821,807 “Robotic Apparatus for Targeting and Producing Deep, Focused Transcranial Magnetic Stimulation.”
Xiao, T. D, Ma, X Q., Zhang, H., Reisner, D. E., Raj, P. M., Wan, L, and R. Tummala, “Magnetic Nanocomposite Paste: An Ideal High-μ, k and Q Nanomaterial for Embedded Inductors in High Frequency Electronic Appls.,” Procs. 9th World Multiconference on Systemics, Cybernetics and Informatics, Jul. 10-13, 2005, Orlando, Fla.
Yoshida, S., Sato, M., Sagawara, E., Shimada, Y., Ono, N., and O. Ito, U.S. Pat. No. 6,972,097, “Composite Magnetic Material and Electromagnetic Interference Suppressor Member Using the Same,” Dec. 6, 2005.

### Summary of Part II

Described herein are Transcranial Magnetic Stimulation (TMS) systems and methods configured to focus the applied magnetic fields generated by one or more primary TMS electromagnets using attractor magnets and/or magnetic concentrators having high-permeability regions. An attractor magnet is typically a secondary, phase-complimentary magnet that is configured to shape the field of the primary TMS electromagnet. A magnetic concentrator typically includes a region of high magnetic permeability region that may shape a TMS magnetic field. Attractor magnets and concentrator devices can be used independently or in combination. The profile of the TMS field can be made narrower or wider than for an unfocused field using these devices, systems and methods.

### Part III: Pitch, Roll, and Yaw Motions for Electromagnet Arrays

Part III describes devices and methods relating generally to the focusing of magnetic fields generated by electromagnets used for Transcranial Magnetic Stimulation.

In general, the devices and systems described herein are configured so that one or more (e.g., an array) Transcranial Magnetic Stimulation (TMS) electromagnets in a TMS system may move in roll, pitch and/or yaw during operation of the TMS system. Any appropriate amount of motion may be used (e.g., small or incremental rotations or large rotations), and the motion may be oscillatory (e.g., repeated) or single-time. In some variations, the roll, pitch and/or yaw motions may be limited so that each electromagnet of the array has a limited range of motion. The TMS electromagnets may be moved in one or any combination of the roll, pitch and yaw motions. In some variations, an entire array of TMS electromagnets is moved through roll, pitch and/or yaw, while in some variations, subsets or individual electromagnets are configured to move in roll, pitch and yaw. These motions may be coordinated by one or more controllers, which may direct the motion, and may coordinate the activity of one or more power supplies, and one or more actuators causing the pitch, roll and yaw movements.

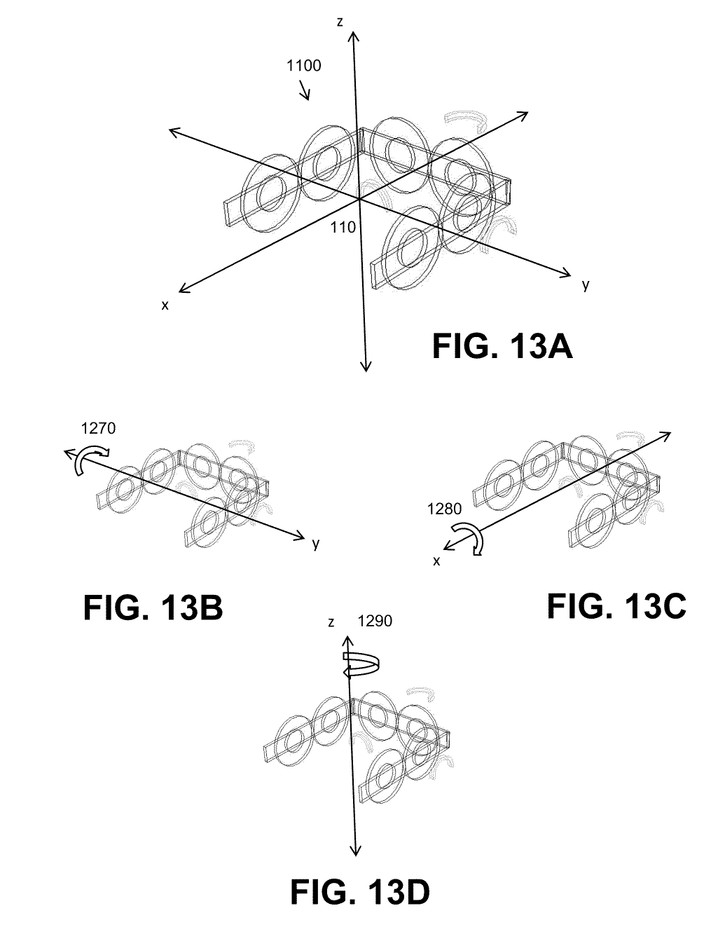

The three movements of pitch, roll and yaw, typically correspond to separate three-dimensional motions around a coordinate system origin. For example, FIG. 13A illustrates one variation of a gantry or frame 1100 having three pairs of TMS electromagnets (“figure-eight” TMS electromagnets). In FIG. 13A, coordinate axes (x, y, z) are overlaid onto the frame 1100. In this example, the intersection of these three axes is an origin 110 that is positioned near the center of the frame, which would be located in the patient's head when the patent is being treated by this device. Pitch, roll and yaw motion of the frame 1100 occurs around this origin 110. FIGS. 13B-13D illustrate motion in pitch, roll and yaw, respectively, of the frame 1100.

Pitch is typically rotation around a lateral or transverse axis, e.g., an axis running from the left to right through the device. As illustrated in FIG. 13B, forward and backward rotation about the y-axis is pitch 1270. Roll is typically rotation around a longitudinal axis, e.g., an axis drawn through the device from front to back. As illustrated in FIG. 13C, roll 1280 is the side-to-side motion about the x-axis. Yaw is typically rotation about a vertical axis, e.g., an axis drawn from top to bottom through the device. As illustrated in FIG. 13D, yaw 1290 is the side-to-side motion about the z-axis.

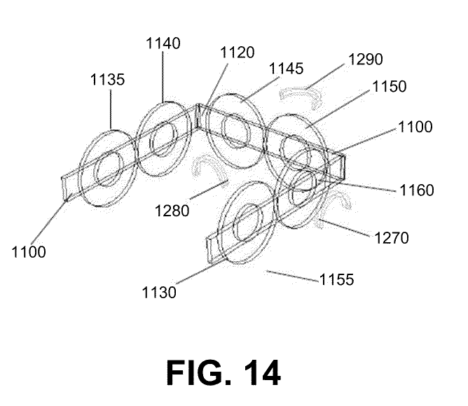

FIG. 14 illustrates one variation of a TMS system including an array of electromagnets that are configured for motion in roll, pitch and/or yaw. In this embodiment the TMS electromagnets are connected to a three-sided frame in which the overall frame/magnet configuration includes a frame 100 consisting of frame sides 1110, 1120, and 1130, and three pairs of coil sets 1135 and 1140, 1145 and 1150, and 1155 and 1160. These coil sets are figure-eight double coils. Such figure-eight double coils for TMS are well known, for instance the 70 mm double-coil configuration from Magstim (e.g., Model 9925, Wales, UK). The electromagnets can be powered by available power sources such as the “Magstim Rapid” (Magstim Ltd., Wales, UK) that provides for pulsed magnetic fields. The system may also include one or more actuators (not shown) for moving the frame in the roll, pitch or yaw directions, as described below, as well as one or more controllers (not shown) for controlling the motion.

As mentioned, the system shown in FIG. 14 is configured so that the frame and attached electromagnets may move in the pitch motion direction 1270, the roll motion direction 1280, and the yaw motion direction 1290. Enough space must be present for any of the motions to ensure that the subject's head is not struck by the device as it moves. Thus, in some variations, the motion in one or more of these directions may be limited.

In operation, a TMS system capable of moving an array of TMS electromagnets in roll, pitch and yaw may be useful for stimulating deep brain regions while minimizing or reducing the stimulation of brain regions located superficial to the deep brain target region (e.g., between the target region and the electromagnets). In particular, systems capable of moving an array of TMS electromagnets in roll, pitch and yaw may be particularly useful for stimulating deep brain target regions that spatially extend along one or more axis within the brain. The system may allow stimulation of a brain region having one or more tracts that extend longitudinally, while limiting the stimulation of superficial regions. For example, the cingulate bundle is a tract that runs anterior-posteriorly in the brain. Targeting of the tracts of the cingualate gyms may be effectively performed by moving (e.g., by oscillating) the frame including an array of TMS electromagnets so that one or more electromagnets follows the path of the cingulated gyms as the frame moves. Pitch (e.g., forward-backward) motion may be especially useful, although combinations of motion including components of yaw and roll may also be used. In practice, multiple motions may be more effective for various coil sizes and shapes since two or more motions can be combined.

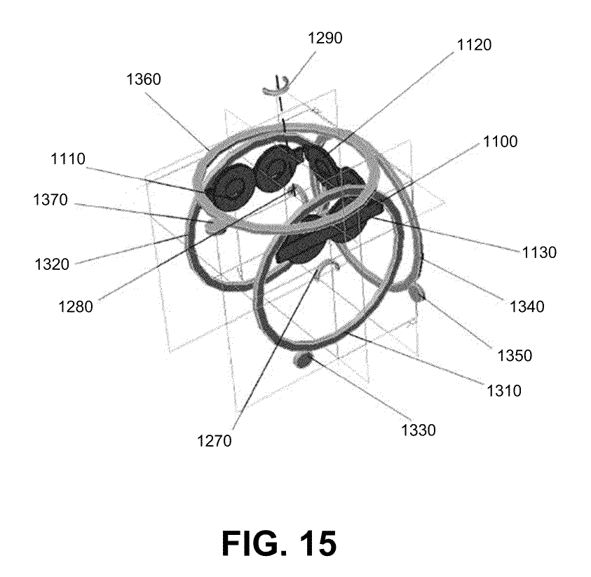

FIG. 15 illustrates another variation of a TMS system configured to move the array of electromagnets. In this example, the array of electromagnets is positioned in frame 1100 that includes frame sides 1110, 1120, and 1130. Three sets of figure-eight double coils attached. In any of the variations described herein, although figure-eight type TMS electromagnets are shown, any appropriate TMS electromagnet may be used. The system shown in FIG. 15 accommodates the pitch motion 1270, the roll motion 1280, and the yaw motion 1290, as shown in FIGS. 13A-13D. Movement of the apparatus in pitch motion 1270 in this example is accomplished by turning rings 1310 and 1320 via drive wheel 1330 with a controlled motor (not shown). Movement of the apparatus in roll motion 1280 is accomplished by turning ring 1340 via drive wheel 1350 with a controlled motor (not shown). Movement of the apparatus in yaw motion is accomplished by turning ring 1360 via drive wheel 1370 with a controlled motor (not shown). Two or more motions can be combined.

The drive wheel and motor referred to above may form part of an actuator for achieving pitch, roll, or yaw. Other actuators may be used, including rotary actuators, step-motors (“steppers”) or other actuators, or the like. Actuators may be coupled to the frame, or (as described in greater detail below) to individual magnets or subsets of magnets in variations in which they are individually movable in pitch, roll and yaw. Thus, any appropriate actuator may be used. A controller may be connected to the actuator to control the motion(s) output by the actuator(s). In some variations the controller includes feedback from the actuators or other portions of the device to indicate the position of the electromagnet(s) being moved.

The examples shown above include systems having an array of electromagnets. In some variations, the systems described herein include only a single TMS electromagnet. Other variations typically include a plurality of electromagnets, forming an array of electromagnets that may be connected to a frame structure (or gantry). As mentioned, an array of electromagnets may have any number of electromagnets. For example, in some variations, the system has a fourth side of the frame (e.g., forming a square that is placed over the subject's head), and includes another pair of TMS electromagnets. In some variations, one or more sides have multiple electromagnets. In some variations, the top of the frame includes one or more electromagnets. For any of these variations, a configuration including one to N magnets is permissible as long as they can be physically accommodated and are of sufficient power to cause the desired effect.

In some variations the frames include different shapes (e.g., round, octagonal. or triangular). Any appropriate shape may be used. The frames may be partially open or enclosed; in some variations, the frame is skeletal and/or wide enough to hold the magnets are without striking the subject. Any of these frames may include one or more electromagnets on a given side. The sizes, numbers, and shapes of magnets may vary. As mentioned above, any appropriate TMS magnet(s) may be used. Frames may include adjustments of dimensions such that different head sizes can be accommodated.

In addition to variations in which the entire array of TMS electromagnets moves in roll, pitch and/or yaw, in some variations one or more subsets or individual magnets in the array moves in roll, pitch and/or yaw. These embodiments are similar to the whole-array variations described above in function and operation.

For example, in one variation each TMS electromagnet has its own, independently functioning, roll, pitch and/or yaw mechanism enabling independent positioning of the electromagnets within the array one or more axes. This may also allow subsets of the TMS electromagnets, or individual TMS electromagnets, to scan across a brain region.

In any of the variations described herein, the position of either the entire array of TMS electromagnets may be moved in roll, pitch and/or yaw at any point before or during the procedure. For example, all or s subset of the electromagnets may be moved prior to starting the stimulation in order to position the electromagnets on one or more targets or to better fit the subject's head. All or a subset of the TMS electromagnets may be moved during stimulation or between stimulation periods; the TMS magnets may be moved either independently of other TMS electromagnets, in a coordinated manner, as part of a synchronized movement (e.g., movement of the entire array via movement of the frame or gantry).

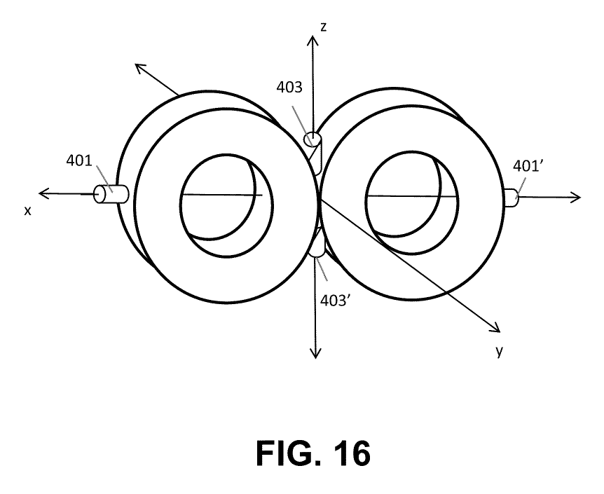

FIG. 16 illustrates a partial view of an individual TMS electromagnet, which may be one of an array of similar TMS electromagnets on a frame that is configured for individual movement in roll, pitch and/or yaw. For example, in FIG. 16, the electromagnet is configured as a figure-8 shaped electromagnet (or pair of electromagnets) that include pivot points in the x-axis 401, 401′, the y-axis (not visible) and the z-axis 403, 403′ directions. These pivots may be connected to one or more actuators that allow the electromagnet to be moved in these axes. Movements in roll (e.g., x axis movement), pitch (y axis movement) and yaw (z axis movement) may be separate or combined.

In variations in which individual TMS electromagnets (or subsets of electromagnets) may move in roll, pitch and yaw, the orientation of individual TMS electromagnets or groups of electromagnets may be altered during the operation of the device. For example, a broader target region may be stimulated by moving one or more TMS electromagnets. In some variations, the TMS electromagnets may be moved so that an electromagnet (or electromagnets) may target a single deep tissue target from different positions relative to the cortical or regions of the brain superficial to the deep brain target. As described above, this may prevent stimulation of the non-target superficial (e.g., cortical) regions.

In some variations, the frame or gantry is configured to move in sections, thereby moving one or subsets of electromagnets. For example one region of the frame may include multiple axes of motion (e.g., pivots) so that the region may be moved in any of pitch, roll, and/or yaw. The axes about which this motion occurs may be centered at any appropriate position, and may be different for different TMS electromagnets or subsets of electromagnets. For example, the center of rotation (the point of intersection of the x, y and z axis) may be centered in the electromagnet, as shown in FIG. 16, or it may centered at a point within the subject's head, as in FIGS. 13A-13D).

In variations of the devices in which one or more individual or groups of TMS electromagnets may be moved either separately or together, one or more controllers may be used to control the motion. For example, a controller may be used to control one or more actuators that move the TMS electromagnet(s) in roll, pitch and/or yaw. The controller may coordinate the motion of different electromagnets, and my include input/feedback from the electromagnets. The controller may also calculate movement pathways in order to prevent collisions between different components of the system and the subject's head or other regions.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, which is set forth in the following claims.

### REFERENCES

M. B. Schneider and D. J. Mishelevich, U.S. patent application Ser. No. 10/821,807 “Robotic Apparatus for Targeting and Producing Deep, Focused Transcranial Magnetic Stimulation.”
D. J. Mishelevich and M. B Schneider, U.S. patent application Ser. No. 11/429,504 “Trajectory-Based Deep-Brain Stereotactic Transcranial Magnetic Stimulation.”

### Summary of Part III

A method for moving an array of electromagnetic coils used for Transcranial Magnetic Stimulation (TMS) in pitch, roll, or yaw modes or two or three of those in combination such that over successive firings of the magnets the neural tissues impacted are not over-stimulated and thus subject to undesirable side effects such as seizures.

### Part IV: Automated Movement of Electromagnets Tracking Eccentricity of the Head

Part IV describes devices and methods that relate generally to moving and positioning electromagnets generating magnetic fields used for Transcranial Magnetic Stimulation.

In general, the systems and devices described herein include a gantry configured to at least partially surround a patient's head, and one or more TMS actuator modules configured to attach to the gantry; the TMS actuator modules include a TMS electromagnet and an actuator that is configured to move the TMS electromagnet towards or away from the subject's head relative to the gantry.

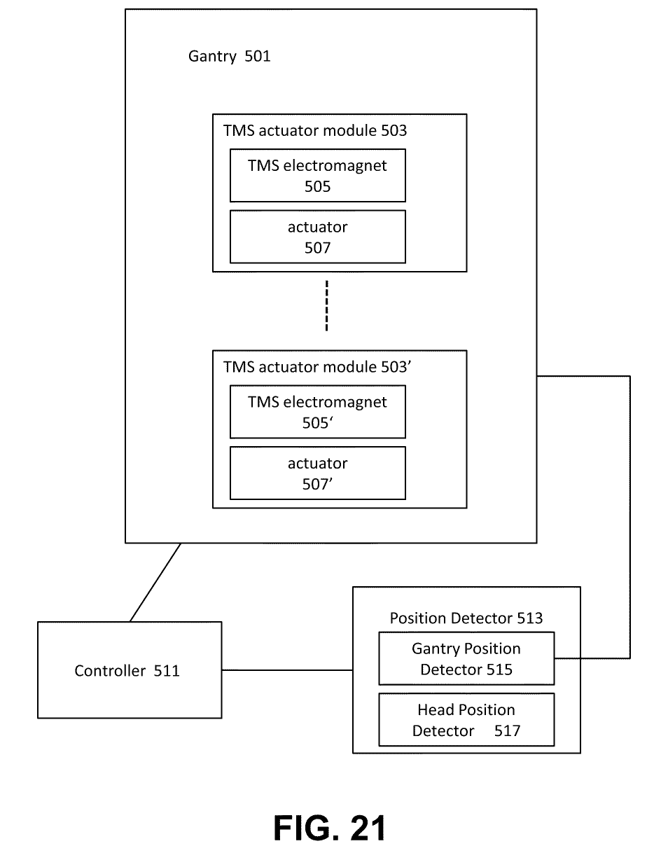

FIG. 21 schematically illustrates one variation of a TMS system and shows the relationship of some of these elements. In this example, a plurality of TMS actuator modules 503, 503′ are positioned on the gantry 501. The gantry may be a frame or track to which the TMS actuator modules are connected. In some variations the TMS actuator modules 503, 503′ are moveably secured to the gantry 501. Thus, the modules 503, 503′ may be moved around the gantry 501. In this variation, the modules may be rotated or moved around the subject's head by moving around the gantry 501. In this variation, the TMS actuator modules may also include a gantry/magnet actuator configured to move the TMS actuator module along the gantry (not shown in FIG. 21). In some variations, the gantry itself may be moved around the subject's head; the modules 503, 503′ may be secured in a fixed position on the gantry, or may be movable as well. An actuator for moving the entire gantry around the subject's head may be referred to as a gantry rotator actuator.

The gantry may be any appropriate shape to fit at least partially around the subject's head, and may be secured to an adjustable mount (e.g., arm or other positioned). Alternatively, the gantry may be rigidly fixed to the system, and the patient may be positioned within it.

In general, the system is configured so that as the modules are moved around the patient's head, the distance between the TMS actuator modules (particularly the TMS electromagnet 505, 505′ of the TMS actuator module) and the patient's head is maintained at a desired separation. For example the distance may be constant, or may be varied or based on a predetermined value given patient anatomy, or based on the target (e.g., the TMS electromagnet may be brought closer when the target is deeper). In the system described herein, the adjustment of the distance between the TMS electromagnet and the patient's head is achieved by the actuator 507, 507′.

The actuator 507, 507′ may be a uniaxial or in/out actuator for moving the TMS electromagnet in/out relative to the gantry or a gantry/magnet actuator for moving the TMS electromagnet around the gantry. In variations in which the actuator is configured to move the electromagnet in and/or out relative to the gantry, the actuator will move the TMS electromagnet closer or further from the patient's head based on either (or both) the position of the TMS actuator module on the gantry or the distance between the TMS electromagnet of the module and the subject's head.

In some variations the TMS actuator module(s) 503, 503′ also includes one or more tilt actuators (not shown) which may also be used to adjust the position of the TMS electromagnet relative to the subject's head. For example, a tilt actuator may be used to aim the TMS electromagnet to a neuronal target as (or after) movement. In some variations the tilt actuator is a limited actuator that may angle the electromagnet slightly (e.g., between −45 and −45 degrees, between 30 and +30 degrees, etc.) in one or two axes. The tilt actuator may be any appropriate actuator configured to tilt the TMS electromagnet as desired. A tilt actuator may be connected to the same controller used to control the primary actuator of the TMS actuator module.

As indicated in FIG. 21, a controller 511 may also be used as part of the system, and may help control the actuator 507, 507′ of the TMS actuator module 503, 503′ to position the TMS electromagnet 505, 505′. The controller may include control logic, and may calculate the distance that the TMS electrode needs to be adjusted by the actuator 507, 507′, and may send instructions to the actuator 507, 507′. The controller may receive input from one or more position detectors 513 (or position sensors). For example, a head-magnet detectors 517 may be included that indicates the distance between the TMS electromagnet and the subject's head. In some variations a gantry position detector 515 be used, which may detect the position of the TMS actuator module on the gantry (in variations which include TMS actuator modules that move relative to the gantry).

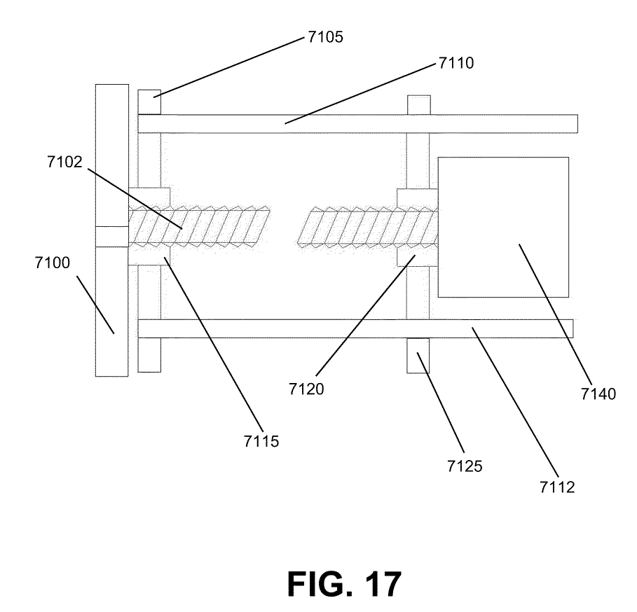

FIG. 17 illustrates one variation of a TMS actuator module. In this variation, the module comprises a TMS electromagnet 7100 and an actuator mechanism 7102. An actuator module moves its associated electromagnet radially in and out (e.g., along one axis). Electromagnet 7100 is moved in and out as threaded rod 7102 (shown divided into two sections in the drawing) moves in and out of collars 7115 and 7120, when driven by linear stepper motor 7140. The apparatus is stabilized on sliding rods 7110 and 7112 that slide through frame elements 7105 and 7125. In another variation, the actuator module uses a similar approach, and incorporates a helically cross-threaded rod that reverses direction automatically at each end of the path, so the motor only needs to rotate in one direction. An example of this helically cross-threaded rod approached in used in some types of fishing reels that have a device that moves back and forth over the spool of the reel to lay down the line in an orderly fashion, first in one direction and then in the other to avoid entanglement of said fishing line. In other variations, the actuator may be a pneumatic actuator, a piezoelectric actuator, a solenoid, etc.

In any of these actuator variations, components of the actuator module are preferably made from non-ferromagnetic materials, particularly the regions nearest the TMS electromagnet. Further, the actuator may be configured so that it does not interfere with the field emitted by the TMS electromagnet.

As mentioned, the extension of the electromagnet from the gantry (e.g., toward or away from the subject's head) may depend on the position of the electromagnet in the orbit. When the position of the gantry where the actuator module is located is far from the patient's head, the electromagnet may be moved in. Conversely, if the position of the gantry where the module is located is close to the patient's head, the electromagnet may be moved out. If multiple electromagnets are simultaneously involved, each may have its own actuator module.

An actuator module, such as the one illustrated in FIG. 17, can be placed in a gantry so that it may ride around a track (e.g., formed from the internal lateral edge or the top edge of the gantry). In another embodiment, the actuator module can be attached to the arm of a robot that is rotating around the head of a patient in a circular or other orbit.

The speed of the actuator in translating the TMS electromagnet (e.g., toward and away from stepper motor 7140) may be increased by including lever arms that translate movements by a plate at the end of the threaded rod into larger movements by the coil. In addition, the actuator may include one or more gears for controlling the motion of the TMS electrode.

As mentioned above, in some variations, the movement of the actuator (e.g., stepper motor 7140) may be determined in part by the position on the gantry. For example, the actuator of the TMS actuator module may be directed by switches that are tripped in anticipation of the module's coils (TMS electromagnet) arriving at a given location with respect to a patient's head. Alternatively, a controller, or device exercising control logic, e.g., a computer running control logic, may direct movements of the actuator (stepper motor 7140).

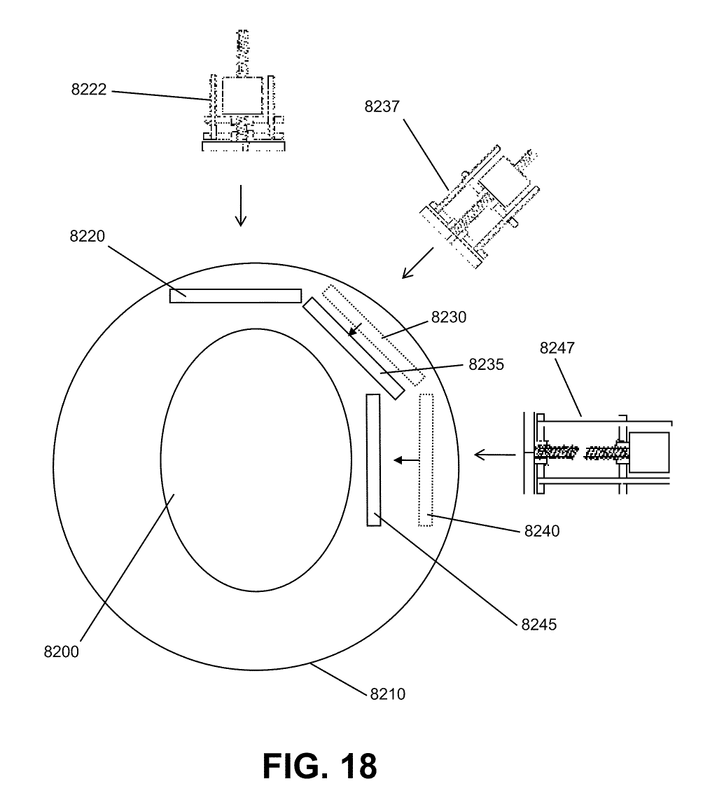

FIG. 18 shows one variation of a circular-gantry embodiment. In this example, the head of the patient (which may be a human or animal subject) 8200 is surrounded by circular gantry 8210. The circular gantry is at some positions closer to the head and sometimes further away. When the position of the TMS electromagnet 8220 is close to the gantry, the associated TMS actuator module 8222 is drawn back. In FIG. 18, for the same of simplicity, the TMS actuator modules are shown removed from the gantry, although the position of the actuator is indicated. Moving clockwise in the diagram, as the actuator module 8237 (including TMS electromagnet 8220, 8230, 8240) circumferentially moves to where what was electromagnet 8220 is now electromagnet 8230, the TMS actuator module moves the electromagnet so it is in position 8235, closer to the head even though the circular gantry is further from the head than it was when the electromagnet was at position 8220. Moving clockwise again, as the actuator module 8247 moves to where what was electromagnet is at position 8240, the TMS actuator module moves the electromagnet so it is now in position 8245, closer to the head even though the circular gantry is further from the head than it was when the electromagnet was at position 8235. Thus, the radial distance of the electromagnet to the head is maintained relatively constant in this example.

Such an embodiment will also apply in cases where the gantry is not circular, but oval (particularly where the shape of the oval of the gantry is not the shape of the oval of the head) or otherwise shaped.

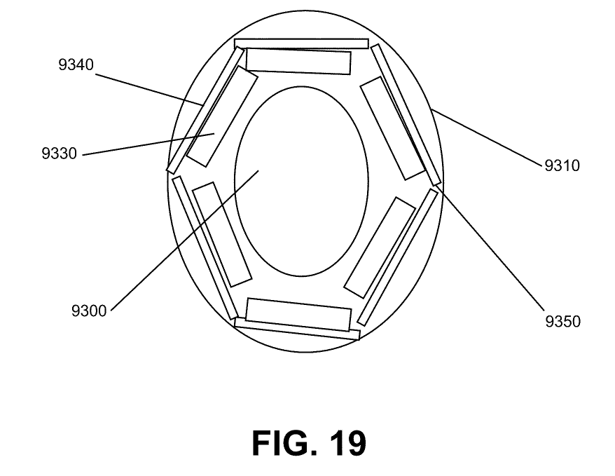

FIG. 19 illustrates a variation having an oval gantry that has one or more electromagnets attached to plates with the plate ends running on a track on the gantry. In FIG. 19, electromagnet(s) 9330 connected to plates 9340 that are configured to run around a gantry 9310 surrounding head 9300. The plates may be connected end to end at points 9350, and the ends of the plates that are attached to the gantry run around the oval gantry, thus allowing radial positioning close or far from the target as appropriate. In one variation, the gantry may also be rotated relative to the center of the patient's head. This may allow the position of a particular electromagnet to be adjusted by rotating both the electromagnet(s) around the gantry, and the gantry around the head. The combined motions of the two may control the separation between the patient's head and the TMS electromagnet. In this variation, the actuator is configured as a gantry actuator, which moves the TMS electromagnet (e.g., attached to plate 9340) around the oval-shaped track, and an additional actuator (not shown) that moves the gantry around the patient's head.

In FIG. 19, there are two types of actuators that may be responsible for adjusting the separation between the TMS electromagnet and the patient's head. First, the TMS actuator module includes an actuator that moves the TMS electrode 9330 (attached to plate 9340) around the oval gantry track. This is a magnet/gantry actuator. Second, the entire gantry (including the track) may be rotated around the subject's head. This second actuator is a gantry rotator actuator. A controller may coordinate the motions of the two actuators. In this example, the TMS actuator module may be thought of as including a single magnet/gantry actuator that moves all of the connected magnets and plates around the oval gantry track.

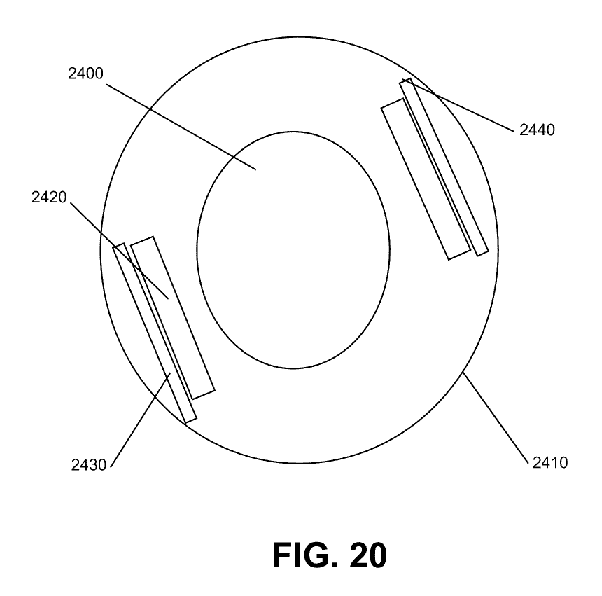

Another oval-gantry embodiment is shown in FIG. 20. In FIG. 20, the oval gantry 2410 surrounds head 2400. Electromagnet(s) 2420 are attached to plates 2430 that are attached at end points 2440 to a track running on the gantry. The track (not shown) is located on the internal lateral surface of the gantry or on the top of the gantry. Again, by combining the motion of the electromagnets 2420 around the oval gantry and the moving the gantry 2410 around the patient's head, the distance between the electromagnets and the patient's head may be controlled.

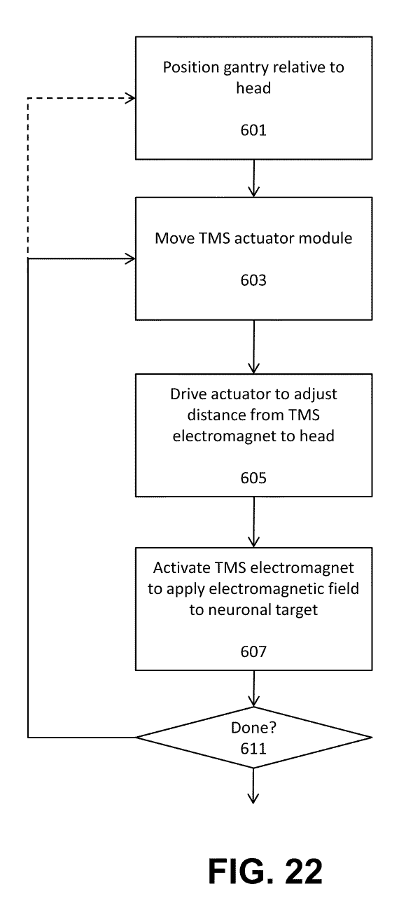

FIG. 22 illustrates one variation of a method of adjusting the distance between a patient and one or more TMS electrodes. In FIG. 22, the first step is to position a gantry relative to the subject's head 601. Next, the TMS actuator module (which includes a TMS electrode and actuator) is moved around the subject's head 603. For example, in the variations shown in FIGS. 17 and 18, in which the modules may move around the gantry or be fixed to the gantry, and include an actuator configured to move the TMS electromagnet in/our relative to the gantry, the module may be moved by a gantry actuator or by moving the entire gantry (with attached module). Similarly, in the variations shown in FIGS. 19 and 20, the TMS actuator module includes a gantry actuator that moves the TMS electrode(s) around the gantry track to a new position. Next, the actuator of the TMS actuator module is driven to adjust the distance between the TMS electromagnet and the subject's head 605. This step may be done concurrently with the step of moving the TMS electrode 603, or it may be done before or after this step. Feedback (e.g., input from one or more detectors) may also be used to help control the movement of the actuator(s). For example, an actuator may extend the TMS electromagnet in/out relative to the gantry. In some variations, an actuator rotates the gantry around the subject's head, changing the separation between the TMS electromagnet and the subject's head. Finally, the TMS electromagnet may be activated 607 to stimulate a region of the subject's brain by TMS. These steps may be repeated 611.

Moving one or more devices in a pathway around a subject's head mirroring the head contours may have uses outside of TMS, as well. For example, devices such as recording electrodes for picking up electronic signals from the brain, sensing coils for detecting magnetic signals emanating from the brain, RF antennae detection radio-frequency signals, or transmitting antenna arrays for stimulating the brain, may all be rotated around a subject's head, and may benefit from the methods and systems described herein. Thus, it is to be understood that these devices and systems may be readily adapted for these uses (e.g., by replacing the TMS electromagnet with one or more of these devices, or other such devices).

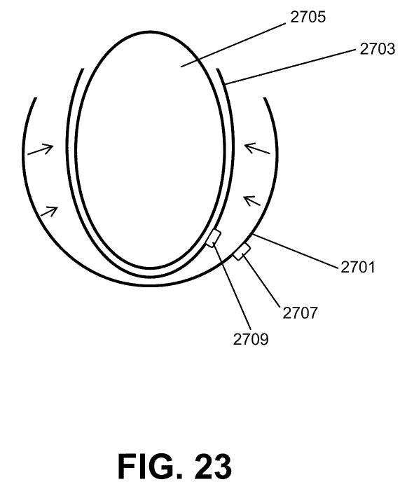

FIG. 23 illustrates another variation of a TMS system including a customizable gantry. In FIG. 23, the gantry initially as a shape 701 that is separated by the patient's head 2705 and does not include a uniform distance between the gantry and the head (or a desired distance). The gantry 2701 may be ‘customized’ by placing it a desired distance x from the patients head, as shown by the arrows in FIG. 23, so that it has the configuration shown 2703. In this example, the gantry may be formed of links or elements that may be bent (e.g., hinged, bendable, etc.) so that they can confirm to the head at a separation distance desired (e.g., a constant distance x). In this example, a single TMS module 2707, 2709 is movably attached to the gantry, as shown. As described for FIGS. 19 and 20, above, the TMS electromagnet may be part of a module (e.g., a base or plate) that is movably attached to the gantry, and may be repositioned by a controller. The gantry may include indicators indicating position, which may be detected, and this information provided to the controller which may use it to control the motion along the gantry.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, which is set forth in the following claims.

### REFERENCES

Avery, D. H., Holtzheimer III, P. E., Fawaz, W., Russo, Joan, Neumaier, J. and Dunner, D. L., Haynor, D. R., Claypoole, K. H., Wajdik, C. and P. Roy-Byrne, “A Controlled Study of Repetitive Transcranial Magnetic Stimulation in Medication-Resistant Major Depression,” Biological Psychiatry, 2005, 59:187-194.
M. B. Schneider and D. J. Mishelevich, U.S. patent application Ser. No. 10/821,807 “Robotic Apparatus for Targeting and Producing Deep, Focused Transcranial Magnetic Stimulation.”

### Summary of Part IV

Methods and System for controlling the motion and position of one or more electromagnets during Transcranial Magnetic Stimulation (TMS) are described. In particular, described herein are systems and methods for rotating one or more electromagnet assemblies around a patient's head in an orbital path that follows the eccentricity of the head to maintain close proximity to the head.

### Part V: Intra-Session Control of Transcranial Magnetic Stimulation

Part V describes devices and methods that relate generally to control of moving, positioning, and activating electromagnets generating magnetic fields used for Transcranial Magnetic Stimulation.

In general, the devices and methods described herein may allow patient feedback based on acute effects during a Transcranial Magnetic Stimulation (TMS) treatment to modify the TMS treatment. In particular, a TMS treatment method begins TMS treatment by applying an initial set of parameters for magnet orientation, power and frequency, and during the course of treatment one or more of these parameters is modified by patient feedback based on the acute experience of the patient during the TMS treatment. Systems for such intra-session control of TMS treatment may include one or more patient inputs, permitting feedback from the patient to modify the ongoing TMS treatment.

For example, described herein are methods including the steps of setting initial configuration parameters for TMS stimulation, stimulating the patient, and receiving direct feedback from the patient based on the acute response of the patient to the TMS treatment, and modifying the TMS treatment based on the feedback. The feedback received from the patient may be control feedback. For example, the patient may manipulate a control or other input for adjusting one or more of the parameters directly. Thus, the patient may tune or adjust a parameter based the patient's experience of one or more acute effects of the TMS therapy.

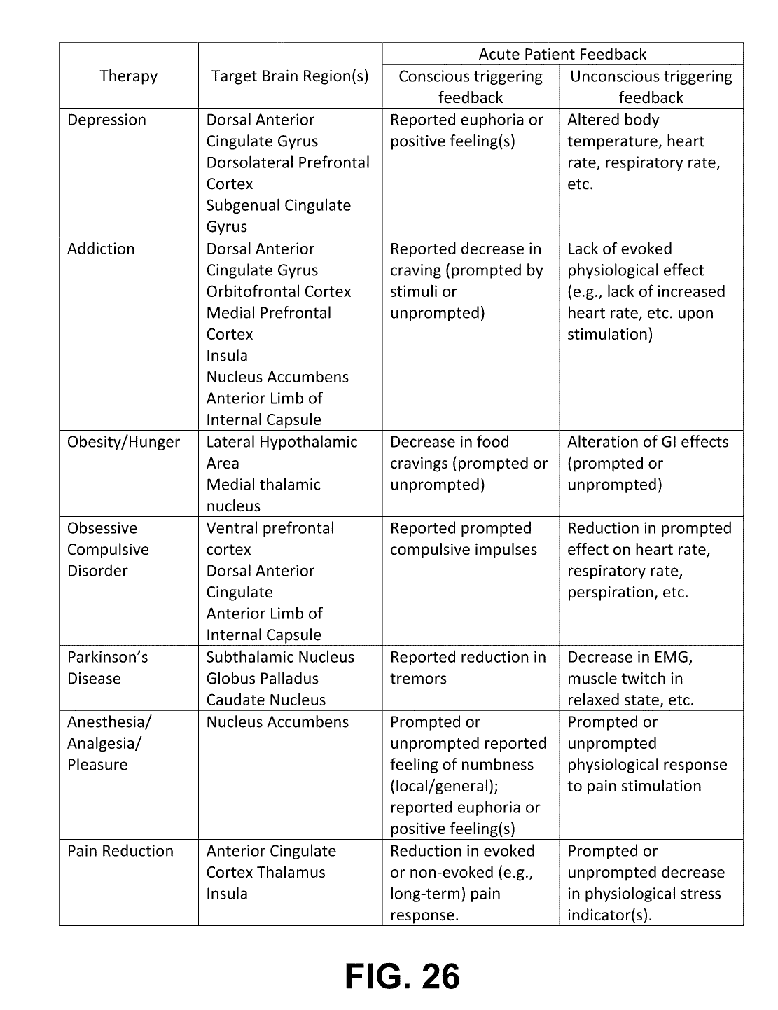

An acute experience of the effect of TMS therapy may be effect that is directly or indirectly associated with the disorder, behavior or condition being treated. For example, FIG. 26 illustrates different therapies that may be treated using TMS, and particularly deep-brain TMS. TMS therapies such as those described may have acute effects that are consciously experienced by the patient, as well as acute effects that are not consciously experienced. As indicated by the last two columns, these conscious and unconscious acute effects may be used as feedback, e.g., triggering feedback, to modify the applied TMS therapy. In particular, the patient may be allowed to adjust a TMS parameter based on the conscious experience of the TMS therapy. For example, a patient being treated for depression may manipulate the position, intensity or frequency of stimulation during the treatment until an acute effect such as a release from the depression or an experience of euphoria is experienced. In some variations the unconscious acute effects of the TMS stimulation may also (or alternatively) be used to adjust one or more parameters of TMS stimulation. In other variations, an unconscious effect of TMS stimulation must be present in order for the system to allow the patient to consciously modify a TMS parameter during treatment. FIG. 26 illustrates various conscious and unconscious effects that may be used to trigger feedback. These examples are not exhaustive, and other effects may be used. Effects that are directly or indirectly correlated with the therapy being applied are of particular interest.

When the triggering feedback is conscious, an alert patient typically manipulates a control to alter one or more stimulation parameter. In practice, the control manipulated may be a handheld control (e.g., button, mouse, joystick, touch screen, etc.), and may be configured so that a patient may manipulate it without moving his or her head or otherwise disturbing the arrangement of the TMS system to the patient's head.

Unconscious triggering feedback may be input from one or more sensors that feed information to the TMS system, including a controller. Thus, monitoring physiological information may be fed back into a controller that adjusts stimulation parameters after analyzing the physiological information. The triggering feedback may be an induced stimulation effect (e.g., identifying an increase or decrease in heart rate, blood pressure, etc.). As used herein, an unconscious triggering feedback measured from the patient is an acute effect that is downstream of the direct effect of the magnetic field applied to the brain region. Thus, the unconscious trigger feedback is not merely an imaging of the brain region being stimulated, showing the effect of TMS on the brain region targeted. Instead, the unconscious trigger feedback results from activation of one or more neural pathways downstream of the stimulated brain region.

A triggering feedback can be triggered as a respond to an inducing stimulus during TMS. For example, during TMS, the patient may be exposed to a stimulus configured to evoke a response that may be modulated by the TMS therapy. The modulation of an acutely evoked response to stimulus may be used to guide feedback for modifying one or more TMS parameters. For example, when treating a disorder such as obesity/overeating, the patient may be exposed to a visual stimulation (e.g., a picture of food) during the TMS therapy. The acute response to this stimulation may be an experience of cravings or an increase in heart rate, etc. The patient may adjust one or more parameters of the TMS therapy until a lessening of this acute response is experienced.

In some variations, the patient triggering feedback is a surrogate experience or an indirect experience, rather than a direct experience. For example, the experience may be an experience/perception that does not directly correlate with the therapy being treated. For example, an experience may be triggered by stimulation of a region of the brain that is nearby (e.g., superficial or adjacent) the target region.

The initial parameters may be set based on a best approximation of the therapeutic target and stimulation protocol. For example, the magnet (or magnets) may be a deep-brain target (e.g., see FIG. 26), and the initial parameters may include a magnetic field intensity that is based on the power applied to TMS electromagnet to stimulate the target without stimulating non-target regions. The frequency of stimulation may also be selected to stimulate (or inhibit) the target. In some variations, the starting parameters may be determined to be within a range of parameters that are calculated to be safe and potentially effective for the target region. This range of values for the parameters may serve as limits to the patient-controlled feedback/inputs.

For example, the initial parameters may include parameters for magnet location and/or orientation, strength of the applied magnetic field, pulse rate, and any other parameters applicable to access the target of interest based on available knowledge.

After receiving patient feedback, one or more parameters may be adjusted by or based on the feedback. As mentioned, the patient in some variations may consciously modify one or more parameters to increase/decrease an acute effect, preferably an effect correlated with the therapy. As part of the therapy or method of performing the therapy, the patient may be instructed on how to adjust/control the TMS stimulation based on a treatment effect. For example, the patient may be told to expect a particular acute effect, and how to modify the therapy based on the acute effect.

After modifying the one or more parameters based on the acute effect, the patient is again (or continues to be) stimulated and allowed to provide additional feedback. In this way a therapeutic response may be optimized. For example, the patient may be treated for acute pain, and during TMS treatment, may modify one or more parameters if the acute pain has not decreased. Feedback inputs may be repeated allowing continuous adjustments to aim, pulse rate, and other parameters. In some variations, a delay or pause may be experienced between the TMS application and the feedback input. Once the optimal effect has been achieved, the values of the parameters may be recorded for use in subsequent sessions. This may help formulate a treatment plan for that patient for that condition. Given the wide range of neurological conditions that are treatable using deep brain TMS, the patient may be potentially treated for multiple conditions that will require multiple configurations, not all of which will have a component of immediate feedback. It is understood that if the patient were being treated for an acute self-limited condition such as acute pain in conjunction with a dental procedure that subsequent treatment sessions may not be required. Alternatively, these optimized conditions may be used as initial parameters that may be later refined, since ‘drift’ of these parameters may be expected.

For any of the methods and devices described herein, suitable magnetic fields can be the type generated by TMS electromagnets such as the double-coil electromagnets available from Magstim, Ltd. (Wales, UK) or those generated by any other type of electromagnet used for TMS combined with pulse-generation systems such as the Rapid², also available from Magstim.

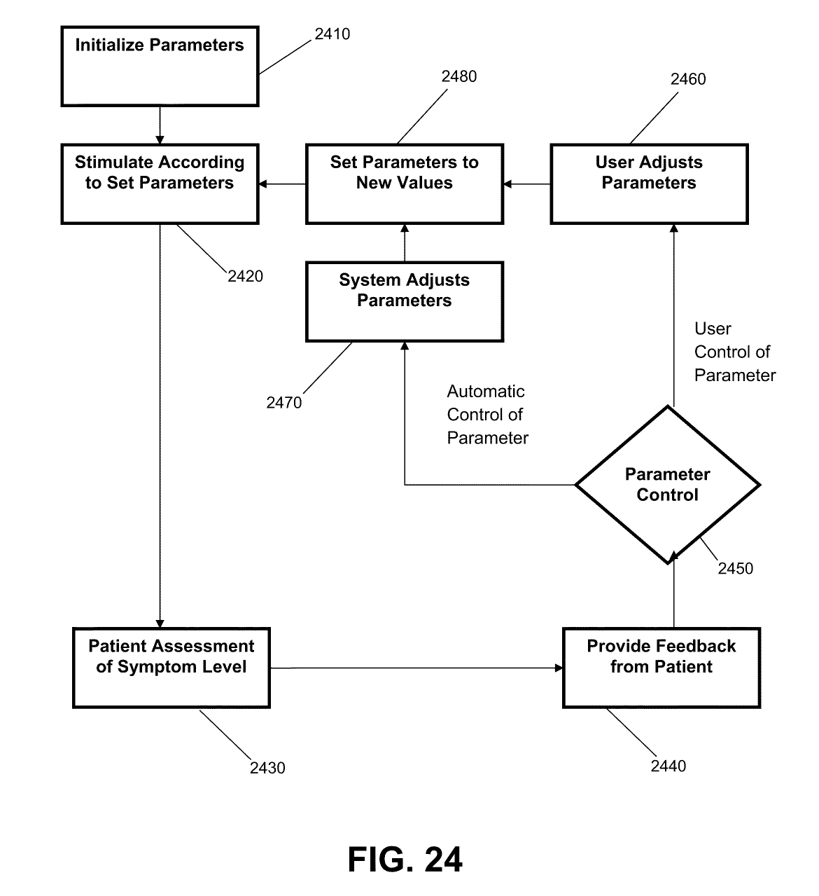

The flow chart FIG. 24 illustrates one example of TMS treatment method including intra-session feedback from the patient. The starting step 2410 initializes the parameters. During the next step 2420, the electromagnet or electromagnets are fired according to the initial set of parameters. Step 2420 is the first step that may be continually in the loop including steps 2420 through 2480. The patient assesses the symptom level (for example level of pain) in step 2430 and provides feedback in step 2440. Step 2440 can involve either a verbal report from the patient or direct patient input in a way (e.g., a Graphic User Interface on a computer) that can be processed automatically. If the parameter control 2450 invokes user parameter control, then the user (physician, nurse, or technician) adjusts parameters in step 2460. If the parameter control 2450 invokes automatic parameter control according to incorporated algorithms, then the system adjusts parameters in step 2470. Whether parameter adjustment occurs in step 2460 or step 2470, the new values are set in step 2480 and the stimulation according to the newly set parameters occurs in step 2420. The loop then continues until the session is completed. The process is applicable irrespective of the type of electromagnet(s) used, whether the electromagnet(s) are moved or not, the type of pulsing, mechanism to vary strength, setting of position or any other parameter.

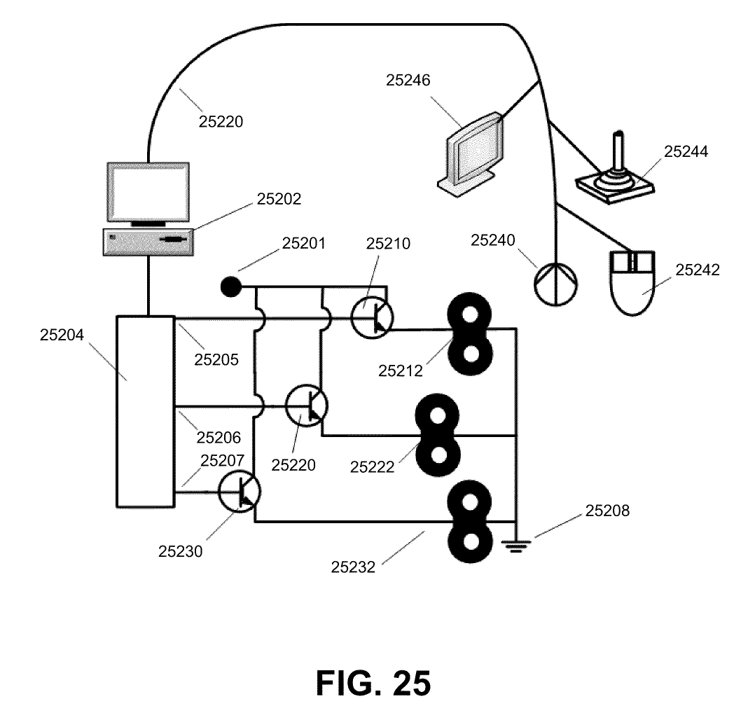

FIG. 25 illustrates one variation of a patient-configurable and optionally self-configuring system, including a control circuit. With this circuit, power is selectively applied to specific coils the array, at specific positions and pulse parameter. Computer 25202 oversees the performance of multi-channel driver 25204, ensuring that pulses are delivered at the right time, and to the proper coils. Multi-channel driver 25204 controls TMS coil 25212 via channel control line 25205, and power transistor 25210. Likewise, TMS coil 25222 is controlled via channel control line 25206 using power transistor 25220, and TMS coil 25232 is controlled via channel control line 25207 using power transistor 25230. The circuits to TMS coils 25212, 25222, and 25232 are completed through ground connection 25208. When power transistors (25210, 25220, 25230) are activated by a corresponding control signal, they activate the corresponding coils by permitting passage of high voltages and currents from the capacitor bank power 25201. In this manner, individual coil circuits may be switched on or off. The coil-activation time can also be controlled by supplying different frequencies of control pulses. Coils may also be moved between physical locations, under the guidance of computer 25202 in accordance with the apparatus described in U.S. patent applications. Ser. No. 11/429,504 and Ser. No. 10/821,807.

Various controls may be used to provide feedback 25220 to computer 25202 regarding which parameters (e.g., coil positions, etc.) and how the parameters should be modified. Such controls may include, for example, transducer 25240, mouse 25242, joystick 25244, or touch-screen computer 25246. In the case of optimization of a treatment for Parkinson's disease, for example, an empiric testing procedure may be conducted with a transducer 25240 in the form of an accelerometer or other motion sensor held in the patient's hand. The patient may then be asked to engage in specific tasks, such as attempting to remain still. Meanwhile, a signal processor examines the signal from the accelerometer, and determines how much tremor is associated with each task, as well as and how accurate and rapid the assigned movements are. During this process, a wide range of candidate stimulus parameter configurations, including position, intensity, and rate for one or more coils may be tested, either by automated or manual empirical processes. The optimal stimulus configuration can be determined empirically, for example, using a hierarchical algorithm to identify the optimal light position configuration for the specific patient. This optimization process can be carried out in an ongoing fashion, by monitoring over a period of days as the patient engages in their normal activities. The optimization process can thus gradually determine the best stimulus profile for the particular patient. At its extremes, all possible parameter configurations of all channels may be automatically tested over a period of time. In a more complex approach, rule-based, or artificial intelligence algorithms may be used to determine optimal parameters for each of the channels.

In the form of an accelerometer, transducer 240 also provides appropriate feedback when the coil is to minimize the amount of motor stimulation that occurs in the context of treatment with the system. By such an approach, the system may learn which positions achieve therapeutic goals without provoking untoward motor movement. One common side effect of rTMS treatment is inadvertent stimulation of the motor cortex, and consequently unintended elicitation of physical movement in the body of the patient. While motor cortex stimulation cannot always be avoided, it is prudent to avoid this phenomenon where possible, and in a manner that does not interfere with the overall treatment plan. For this purpose, inadvertent movement, as signaled by a transducer, may constitute feedback in the context of the present invention.

Various other input and testing procedures can be used depending upon the specific problem being treated. The patient's preference may be entered into a computer via text, graphic user interface, and/or device such as a mouse, track pad, trackball or joystick, or 3D optical tracking device. Various other brain-machine interfaces may also be used as part of the testing and optimization routine. It will be appreciated that the optimization process may be conducted in an open-loop (manual device configuration) or closed loop (fully automated device configuration) manner.

If appropriate measures of patient performance (for example freedom from tremor as measured by an accelerometer) are detected, this information can be automatically fed back to computer 25202 for storage in a database. Computer 25202 can use the stored information in accordance with algorithms and artificial intelligence methods to determine a suitable stimulation solution using driver 25204.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, which is set forth in the following claims.

### REFERENCES

Avery, D. H., Holtzheimer III, P. E., Fawaz, W., Russo, Joan, Neumaier, J. and Dunner, D. L., Haynor, D. R., Claypoole, K. H., Wajdik, C. and P. Roy-Byrne, “A Controlled Study of Repetitive Transcranial Magnetic Stimulation in Medication-Resistant Major Depression,” Biological Psychiatry, 2005; 59:187-194.
Bohning, D. and M. George, “Method, Apparatus, and System for Automatically Positioning a Probe or Sensor,” U.S. patent application Ser. No. 10/991,129.
Fox, P. and J. Lancaster, “Apparatus and Methods for Delivery of Transcranial Magnetic Stimulation,” U.S. Pat. No. 7,087,008.
George, M. S., Kozel, F. A., and D. E. Bohning, “Functional Magnetic Resonance Imaging Guided Transcranial Magnetic Stimulation Deception Inhibitor,” U.S. patent application Ser. No. 10/521,373.
Ives, J. R., Pascual-Leone, A. and Q. Chen, “Method and Apparatus for Monitoring a Magnetic Resonance Image During Transcranial Magnetic Stimulation,” U.S. Pat. No. 6,198,958.
Ives, J. R. and A. Pascual-Leone, “Method and Apparatus for Recording an Electroencephalogram During Transcranial Magnetic Stimulation,” U.S. Pat. Nos. 6,266,556 and 6,571,123.
Mayberg, H. S., Lazano, A. M., Voon, V., McNeely, H. E., Seminowicz, D., Hamani, C., Schwalb, J. M., and S. H. Kennedy, “Deep Brain Stimulation for Treatment-Resistant Depression. Neuron,” 2005 Mar. 3; 45:651-60.
D. J. Mishelevich and M. B. Schneider, “Trajectory-Based Transcranial Magnetic Stimulation,” U.S. patent application Ser. No. 11/429,504.
Riehl, M. E., “Determining Stimulation Levels for Transcranial Magnetic Stimulation,” U.S. Pat. No. 7,104,947.
M. B. Schneider and D. J. Mishelevich, “Robotic Apparatus for Targeting and Producing Deep, Focused Transcranial Magnetic Stimulation,” U.S. patent application Ser. No. 10/821,807.
Tanner, P., “Methods and Devices for Transcranial Magnetic Stimulation and Cortical Cartography,” U.S. Pat. Nos. 6,830,544 and 7,239,910.
Tanner, P., Hartlep, A., Wist, H., Wendicke, K. and T. Weyh, “Method and Device for Transcranial Magnetic Stimulation,” U.S. Pat. No. 7,008,370.

### Summary of Part V

Described herein are methods for controlling Transcranial Magnetic Stimulation during or within a session, where direct immediate patient reported feedback is utilized to assess the effect and optimize the treatment in real time. These methods may be applicable to superficial repetitive Transcranial Magnet Stimulation (rTMS) or deep-brain stereotactic Transcranial Magnetic Stimulation (sTMS). Examples of therapies that may benefit from these methods include TMS treatment of: acute pain (e.g., during dental procedures or bunionectomies), depression, or Parkinson's Disease, to name only a few. TMS systems and devices including or more patient inputs that may be used to perform these methods are also described.

We claim:

1. An arrangement of two or more therapeutic magnetic coils in series, in which the positive and negative poles are passed through a bridge rectifier, whereby the electrical currents in each of the coils are substantially in single directions.

2. An arrangement of two or more therapeutic magnetic coils, in which the phase of the electrical current passing through the coils has been matched to the polarity and position of the coils such that matched coil polarity orientation and matched B-field direction yields summation.

3. An arrangement of two or more therapeutic magnetic coils, in which the phase of the electrical current passing through the coils has been matched to the polarity and position of the coils such that opposite coil polarity orientation and opposite B-field direction yields summation.

4. An arrangement of two or more therapeutic magnetic coils, in which the phase of the electrical current passing through the coils has been matched to the polarity and position of the coils such that matched phase and opposite direction yields cancellation.

5. An arrangement of two or more therapeutic magnetic coils, in which the phase of the electrical current passing through the coils has been matched to the polarity and position of the coils such that opposite phase and matched direction yields cancellation.

6. A Transcranial Magnetic Stimulation system for stimulating a subject's neuronal tissue, the system including:
a primary electromagnet configured to apply Transcranial Magnetic Stimulation to the subject; and
an attractor magnet, wherein the attractor magnet is configured to be isolated from the primary electromagnet and to apply an electromagnetic field that is opposite in polarity in a phase-complementary manner at any given time with the primary electromagnet magnet.

7. The system of claim 6, further comprising a controller configured to coordinate activation of the primary electromagnet and the attractor magnet.

8. The system of claim 6, further comprising a controller configured to coordinate the positions of the attractor magnet and the primary electromagnet relative to a neuronal target.

9. The system of claim 6, wherein the attractor magnet is configured to be positioned opposite the primary electromagnet by 180 degrees.

10. The system of claim 6, wherein the attractor magnet is configured to be positioned opposite the primary electromagnet by 90 degrees.

11. The system of claim 6, further comprising at least one magnetic concentrator comprising a shaped region of high magnetic permeability, wherein the concentrator is configured to modify the Transcranial Magnetic Stimulation applied by the primary electromagnet.

12. The system of claim 11, wherein the magnetic concentrator is isolated from the primary electromagnet.

13. The system of claim 6, further comprising a second primary electromagnet configured to apply Transcranial Magnetic Stimulation to the subject.

14. A Transcranial Magnetic Stimulation system for stimulating a subject's neuronal tissue, the system including:
a primary electromagnet configured to apply Transcranial Magnetic Stimulation to the subject; and
a magnetic concentrator comprising a shaped region of high magnetic permeability, wherein the concentrator is configured to modify the Transcranial Magnetic Stimulation applied by the primary electromagnet, further wherein the magnetic concentrator is configured to be positioned on or within the subject separately from the primary electromagnet.

15. The system of claim 14, further comprising an attractor magnet, wherein the attractor magnet is configured to be positioned opposite the primary electromagnet and to apply an electromagnetic field that is opposite in polarity in a phase-complementary manner at any given time with the primary electromagnet magnet.

16. The system of claim 14, further comprising a controller configured to coordinate the positions of the primary electromagnet and the magnetic concentrator relative to a neuronal target.

17. The system of claim 14, wherein the magnetic concentrator is disposable or single-use.

18. The system of claim 14, wherein the magnetic concentrator is configured to be applied to the outside of the patient's head.

19. The system of claim 14, wherein the magnetic concentrator is configured to be inserted in a subject's body.

20. The system of claim 14, wherein the magnetic concentrator is configured to be held in the patient's mouth.

21. The system of claim 14, further comprising a plurality of magnetic concentrators.

22. A Transcranial Magnetic Stimulation method for stimulating a neuronal target tissue, the method comprising:
selecting the neuronal target;
positioning a primary electromagnet to apply electromagnetic energy to the target;
positioning an attractor magnet opposite the primary electromagnet;
emitting an electromagnetic field from the primary electromagnet; and
emitting an electromagnetic field from the attractor magnet that is opposite in polarity in a phase-complementary manner at any given time with the electromagnetic field emitted from the primary electromagnet magnet, so that the magnetic field applied to the neuronal target from the primary electromagnet is focused by the electromagnet field from the attractor magnet.

23. The method of claim 22, further comprising positioning a magnetic concentrator on or within the subject to enhance the electromagnetic energy applied to the target from the primary electromagnet.

24. The method of claim 22, further comprising positioning a magnet concentrator on or within the subject to shield a region of the subject from the electromagnetic energy applied to the target.

25. The method of claim 22, further comprising determining the energy applied to the primary electromagnet based on the positions of the target, primary electromagnet and attractor electromagnet.

26. A Transcranial Magnetic Stimulation method for stimulating a neuronal target tissue, the method comprising:
selecting the neuronal target;
positioning a primary electromagnet to apply electromagnetic energy to the target;
positioning a magnetic concentrator on or within the subject to enhance the electromagnetic energy applied to the target from the primary electromagnet; and
emitting an electromagnetic field from the primary electromagnet so that the emitted electromagnetic field is altered by the magnetic concentrator.

27. The method of claim 26, wherein the step of positioning the magnetic concentrator comprises positioning the concentrator within the subject's body.

28. The method of claim 26, wherein the step of positioning the magnetic concentrator comprises positioning a disposable magnetic concentrator.

29. The method of claim 26, wherein the step of positioning the magnetic concentrator comprises applying the magnetic concentrator comprises applying the magnetic concentrator to the subject's head.

30. The method of claim 26, further comprising determining the energy applied to the primary electromagnet based on the position of the primary electromagnet, the target and the magnetic concentrator.

31. A Transcranial Magnetic Stimulation method for stimulating a neuronal target tissue, the method comprising:
selecting the neuronal target;
positioning a primary electromagnet to apply electromagnetic energy to the target;
positioning a magnetic concentrator on or within the subject to shield a region of the subject from the electromagnetic energy applied to the target by the primary electromagnet; and
emitting an electromagnetic field from the primary electromagnet so that the emitted electromagnetic field is altered by the magnetic concentrator.

32. A Transcranial Magnetic Stimulation system for stimulating a neuronal target, the system comprising:
a frame assembly configured to be placed adjacent to a subject's head;
at least one Transcranial Magnetic Stimulation electromagnet supported by the frame;
a roll actuator configured to move the Transcranial Magnetic Stimulation electromagnet in roll;
a pitch actuator configured to move the Transcranial Magnetic Stimulation electromagnet in pitch; and
a yaw actuator configured to move the Transcranial Magnetic Stimulation electromagnet in yaw.

33. The system of claim 32, further comprising a controller communicating with the roll, pitch and yaw actuators, wherein the controller is configured to control movement of the Transcranial Magnetic Stimulation electromagnet in roll, pitch and yaw.

34. The system of claim 32, further comprising a plurality of Transcranial Magnetic Stimulation electromagnets supported by the frame.

35. The system of claim 34, wherein the actuators are configured to move the plurality of Transcranial Magnetic Stimulation electromagnets by moving the frame assembly in roll, pitch and yaw.

36. The system of claim 34, wherein the each of the Transcranial Magnetic Stimulation electromagnets is configured to be independently moveable in roll, pitch and yaw with respect to the other Transcranial Magnetic Stimulation electromagnets.

37. A Transcranial Magnetic Stimulation system for stimulating a subject's neuronal system, the system comprising:
a frame assembly;
an array of Transcranial Magnetic Stimulation electromagnets supported by the frame; and
a plurality of actuators, wherein the actuators are configured to move the array of Transcranial Magnetic Stimulation electromagnets in roll, pitch and yaw.

38. The system of claim 37, further comprising a controller communicating with the plurality of actuators and configured to control movement of the Transcranial Magnetic Stimulation electromagnet in roll, pitch and yaw.

39. The system of claim 37, wherein the plurality of actuators are configured to simultaneously move the entire array of Transcranial Magnetic Stimulation electromagnets in roll, pitch and yaw by moving the frame assembly.

40. The system of claim 37, wherein the each of the Transcranial Magnetic Stimulation electromagnets is configured to be independently moveable in roll, pitch and yaw with respect to the other Transcranial Magnetic Stimulation electromagnets.

41. A Transcranial Magnetic Stimulation system for stimulating a neuronal target, the system comprising:
a frame assembly;
a plurality of Transcranial Magnetic Stimulation electromagnets supported by the frame;
a plurality of actuators; and
a controller communicating with the plurality of actuators,
wherein the Transcranial Magnetic Stimulation electromagnets is configured to be moved in roll, pitch and yaw by the actuators, so that each Transcranial Magnetic Stimulation electromagnet or a subset of the Transcranial Magnetic Stimulation electromagnets may be moved relative to the other Transcranial Stimulation electromagnets.

42. A Transcranial Magnetic Stimulation method for stimulating a neuronal target tissue, the method comprising:
positioning a frame including at least one Transcranial Magnetic Stimulation electromagnet around a subject's head; and
moving the Transcranial Magnetic Stimulation electromagnet in roll, pitch and/or yaw.

43. The method of claim 42, wherein the step of moving the Transcranial Magnetic Stimulation electromagnet comprises moving the frame to which the Transcranial Magnetic Stimulation electromagnet is attached.

44. The method claim 42, wherein the step of moving the Transcranial Magnetic Stimulation electromagnet comprises moving the Transcranial Magnetic Stimulation electromagnet relative to another Transcranial Magnetic Stimulation electromagnet included on the frame.

45. The method of claim 42, further comprising activating the Transcranial Magnetic Stimulation electromagnet prior to moving the Transcranial Magnetic Stimulation electromagnet.

46. The method of claim 42, wherein the step of moving the Transcranial Magnetic Stimulation electromagnet comprises activating one or more actuators to move the Transcranial Magnetic Stimulation electromagnet.

47. The method of claim 42, further comprising the step of moving the Transcranial Magnetic Stimulation electromagnet prior to activating the Transcranial Magnetic Stimulation electromagnet.

48. The method of claim 42, further wherein the step of moving the Transcranial Magnetic Stimulation electromagnet comprises moving the Transcranial Magnetic Stimulation electromagnet in roll pitch and/or yaw relative to the frame.

49. A Transcranial Magnetic Stimulation method for stimulating a neuronal target tissue, the method comprising:
positioning a frame including a plurality of Transcranial Magnetic Stimulation electromagnet around a subject's head; and
moving one or a subset of the Transcranial Magnetic Stimulation electromagnets in roll, pitch and/or yaw relative to the other Transcranial Magnetic Stimulation electromagnet(s).

50. The method of claim 49, further comprising activating one or more of the Transcranial Magnetic Stimulation electromagnets prior to the step of moving one or a subset of the Transcranial Magnetic Stimulation electromagnets.

51. A Transcranial Magnetic Stimulation (TMS) system comprising:
a gantry configured to at least partially encircle a patient's head; and
a TMS actuator module connected to the gantry, wherein the TMS actuator module comprises
a TMS electromagnet and
an actuator for moving the TMS electromagnet in or out relative to the gantry;
wherein the TMS actuator module is further configured to move the TMS electromagnet to adjust the distance between the TMS electromagnet and the surface of the patient's head.

52. The system of claim 51, further comprising a controller configured to determine the position of the TMS actuator module and to instruct the actuator of the TMS actuator module to adjust position of the TMS electromagnet relative to a patient's head.

53. The system of claim 51, wherein the actuator comprises a uniaxial actuator.

54. The system of claim 51, further comprising a plurality of TMS actuator modules, wherein each TMS actuator module comprises a TMS electromagnet and an actuator for moving the TMS electromagnet in or out relative to the gantry.

55. The system of claim 51, wherein the TMS actuator module is configured to move along the gantry and comprises a gantry/magnet actuator configured to move the TMS actuator module along the gantry.

56. The system of claim 51, wherein the TMS actuator module is secured in position on the gantry.

57. The system of claim 51, further comprising a position detector configured to determine the position of the TMS actuator module on the gantry.

58. The system of claim 51, further comprising a position detector configured to determine the position of the TMS electromagnet relative to a patient's head.

59. The system of claim 51, wherein the TMS actuator module comprises a tilt actuator configured to adjust the angle of the TMS electromagnet relative to a patient's head.

60. The system of claim 51, wherein the gantry comprises position indicators.

61. The system of claim 51, wherein the gantry is selected from the group consisting of: a circular gantry, an oval gantry, a semi-circular gantry.

62. A Transcranial Magnetic Stimulation (TMS) system comprising:
a gantry;
a gantry rotator actuator configured to rotate the gantry around a subject's head;
a TMS actuator module on the gantry, wherein the TMS actuator module comprises
a TMS electromagnet, and
a gantry/magnet actuator configured to move the TMS electromagnet along the gantry; and
a controller configured to determine the position of the TMS electromagnet relative to the patient's head and to coordinate the motion of the gantry rotator actuator and the actuator of the TMS actuator module to adjust the distance between the TMS electromagnet and the surface of the patient's head.

63. The system of claim 62, wherein the gantry is elliptical.

64. A Transcranial Magnetic Stimulation (TMS) method, the method comprising:
moving a TMS actuator module along a gantry, wherein the TMS actuator module comprises a TMS electromagnet and an actuator configured to adjust the position of the TMS electromagnet relative to the gantry; and
adjusting the distance between the TMS electromagnet and the patient's head using the actuator.

65. The method of claim 64, wherein the step of adjusting the distance between the TMS electromagnet and the patient's head comprises maintaining a relatively constant distance between the TMS electromagnet and the patient's head as the TMS actuator module is moved along the gantry.

66. The method of claim 64, further comprising rotating the gantry around the subject's head.

67. The method of claim 64, further comprising determining the position of the TMS electromagnet relative to the patient's head.

68. The method of claim 64, wherein the step of adjusting the distance between the TMS electromagnet and the patient's head comprises determining the distance between the TMS electromagnet and the subject's head and controlling the actuator to adjust the distance of the TMS electromagnet based on this distance.

69. A Transcranial Magnetic Stimulation (TMS) method, the method comprising:
positioning a gantry relative to a subject's head;
moving a TMS actuator module along a gantry, wherein the TMS actuator module comprises a TMS electromagnet and an actuator configured to move the TMS electromagnet towards or away from a patient's head relative to the gantry;
determining the position of the TMS electromagnet relative to the patient's head;
adjusting the distance between the TMS electromagnet and the patient's head using the actuator; and
activating the TMS electromagnet to apply an electric field to a neuronal target.

70. The method of claim 69, wherein the step of adjusting the distance between the TMS electromagnet and the patient's head comprises maintaining a relatively constant distance between the TMS electromagnet and the patient's head as the TMS actuator module is moved along the gantry.

71. A Transcranial tracking system, the system comprising:
a gantry configured to at least partially encircle a patient's head; and
an actuator module connected to the gantry, wherein the actuator module comprises
an energy source and
an actuator for moving the energy source in or out relative to the gantry;
wherein the actuator module is further configured to move the energy source to adjust the distance between the energy source and the surface of the patient's head.

72. The system of claim 71, wherein the energy source is selected from the group consisting of: light, radio frequency, acoustic, and, thermal energy sources.

73. A Transcranial tracking system, the system comprising:
a gantry configured to at least partially encircle a patient's head; and
an actuator module connected to the gantry, wherein the actuator module comprises a sensor and
an actuator for moving the sensor in or out relative to the gantry;
wherein the actuator module is further configured to move the sensor to adjust the distance between the energy source and the surface of the patient's head.

74. A patient-configurable Transcranial Magnetic Stimulation (TMS) method that allows a patient to dynamically modify the TMS while a TMS procedure is being performed, the method comprising:
applying Transcranial Magnetic Stimulation to a first site in the patient's brain, at a first magnetic field intensity and a first stimulation frequency;
changing one or more of the site, intensity or the frequency of the TMS stimulation based on input from the patient, wherein the patient changes one or more of the site, intensity or frequency of the TMS stimulation based on the patient's experience of the applied TMS stimulation; and
applying Transcranial Magnetic Stimulation to the patient at the new site, intensity or frequency of TMS stimulation.

75. The method of claim 74, further comprising providing a stimulus to prompt a patient experience that is modified during the TMS procedure.

76. The method of claim 75, wherein the stimulus comprises a visual stimulus.

77. The method of claim 75, wherein the stimulus comprises a tactile stimulus.

78. The method of claim 74, wherein the step of changing one or more of the site, intensity or the frequency of the TMS stimulation comprises allowing the patient to manipulate a handheld control to alter one or more of the site, intensity or frequency of the TMS stimulation.

79. The method of claim 78, wherein the patient may alter the site, intensity or frequency of the TMS stimulation only within a predetermined range for each of the site, intensity or frequency.

80. The method of claim 74, wherein the step of changing one or more of the site, intensity or the frequency of the TMS stimulation is performed while applying Transcranial Magnetic Stimulation to the patient.

81. A patient-configurable Transcranial Magnetic Stimulation (TMS) method that allows a patient to dynamically modify the TMS while a TMS procedure is being performed, the method comprising:
positioning a plurality of TMS electromagnets to apply electromagnetic energy to a deep brain target site;
applying TMS to the target site at a magnetic field intensity and a stimulation frequency;
enabling the patient to change one or more of the position of the TMS electromagnet, the intensity of the TMS stimulation, or the frequency of the TMS stimulation based the patient's experience of the applied TMS stimulation; and
applying Transcranial Magnetic Stimulation to the patient at the changed position of the TMS electromagnet, intensity of the TMS stimulation, or frequency of TMS stimulation.

82. The method of claim 81, further comprising providing a stimulus to prompt a patient experience that is modified during the TMS procedure.

83. The method of claim 82, wherein the stimulus comprises a visual stimulus.

84. The method of claim 82, wherein the stimulus comprises a tactile stimulus.

85. The method of claim 81, wherein the step of enabling the patient to change one or more of the position of the TMS electromagnet, the intensity of the TMS stimulation, or the frequency of the TMS stimulation comprises allowing the patient to manipulate a handheld control.

86. A system for applying Transcranial Magnetic Stimulation (TMS), the system comprising:
at least one TMS electromagnet configured to apply TMS to a site in a patient's brain;
a controller configured to control the TMS electromagnet to apply TMS to the site in a patient's brain at a magnetic field intensity and a frequency of stimulation; and
a patient feedback input connected to the controller, configured to allow the patient to adjust one or more of the site of application of the TMS in the patient's brain, the magnetic field intensity of the applied TMS, or the frequency of the TMS stimulation during a TMS procedure on the patient.

87. The system of claim 86, wherein the at least one TMS electromagnet comprises a plurality of TMS electromagnets configured to be positioned to apply TMS to a site in a patient's brain at a magnetic field intensity and a frequency of stimulation.

88. The system of claim 86, wherein the controller is configured to coordinate the stimulation applied by a plurality of TMS electromagnets to apply TMS to a deep brain target.

89. The system of claim 86, wherein the patient feedback input comprises a joystick.

90. The system of claim 86, wherein the patient feedback input comprises a mouse.

91. The system of claim 86, wherein the controller is configured to limit the adjustment of the site of application of the TMS in the patient's brain, the magnetic field intensity of the applied TMS, and the frequency of the TMS stimulation by the patient feedback input so that these parameters remain within a predetermined range of values.

92. A system for applying Transcranial Magnetic Stimulation (TMS), the system comprising:
a plurality of TMS electromagnets configured to apply TMS to a deep brain target site in a patient's brain;
a controller configured to control the plurality of TMS electromagnets to apply TMS to the target site in the patient's brain at a magnetic field intensity and a frequency of stimulation; and
at least one patient feedback input configured to allow the patient to adjust one or more of the site of application of the TMS in the patient's brain, the magnetic field intensity of the applied TMS, or the frequency of the TMS stimulation during a TMS procedure on the patient.

93. The system of claim 92, wherein the patient feedback input is selected from the group consisting of: a joystick, a mouse, a touch screen, and a motion sensor.
